SEQUENCE LISTING (7 sequences)

SEQ ID NO: 1 - protein, 9 aa, Artificial Sequence
  Description of Artificial Sequence Synthetic peptide
CSPGAKVRC

SEQ ID NO: 2 - DNA, 27 bp, Artificial Sequence
  Description of Artificial Sequence Synthetic oligonucleotide
tgctctccgggggcgaaagtcaggtgc

SEQ ID NO: 3 - DNA, 2064 bp, Artificial Sequence
  Description of Artificial Sequence Synthetic polynucleotide
gctagcatgtgcctctctccggttaaaggcgcaaagctcatcctgatctttctgttcctaggagccgttcagtccaatgcagcgcgatgctctccgggggcgaaagtcaggtgcgctcgtggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatgagctcagcacccacaaagctagctggagcggaggcttgatagttaatttgacagattcaaagggtacttgcctttatgcagaatgggagatgaatttcacaataacatatgaaactacaaaccaaaccaataaaactataaccattgcagtacctgacaaggcgacacacgatggaagcagttgtggggatgaccggaatagtgccaaaataatgatacaatttggattcgctgtctcttgggctgtgaattttaccaaggaagcatctcattattcaattcatgacatcgtgctttcctacaacactagtgatagcacagtatttcctggtgctgtagctaaaggagttcatactgttaaaaatcctgagaatttcaaagttccattggatgtcatctttaagtgcaatagtgttttaacttacaacctgactcctgtcgttcagaaatattggggtattcacctgcaagcttttgtccaaaatggtacagtgagtaaaaatgaacaagtgtgtgaagaagaccaaactcccaccactgtggcacccatcattcacaccactgccccgtcgactacaactacactcactccaacttcaacacccactccaactccaactccaactccaaccgttggaaactacagcattagaaatggcaatactacctgtctgctggctaccatggggctgcagctgaacatcactgaggagaaggtgcctttcatttttaacatcaaccctgccacaaccaacttcaccggcagctgtcaacctcaaagtgctcaacttaggctgaacaacagccaaattaagtatcttgactttatctttgctgtgaaaaatgaaaaacggttctatctgaaggaagtgaatgtctacatgtatttggctaatggctcagctttcaacatttccaacaagaaccttagcttctgggatgcccctctgggaagttcttatatgtgcaacaaagagcaggtgctttctgtgtctagagcgtttcagatcaacacctttaacctaaaggtgcaaccttttaatgtgacaaaaggacagtattctacagctgaggaatgtgctgctgactctgacctcaactttcttattcctgttgcagtgggtgtggccttgggcttccttataattgctgtgtttatatcttacatgattggaagacggaaaagtcgtactggttatcagtctgtccaccaccaccaccaccactaagaattc

SEQ ID NO: 4 - DNA, 81 bp, Artificial Sequence
  Description of Artificial Sequence Synthetic oligonucleotide
gctagcatgtgcctctctccggttaaaggcgcaaagctcatcctgatctttctgttcctaggagccgttcagtccaatgca

SEQ ID NO: 5 - DNA, 2037 bp, Artificial Sequence
  Description of Artificial Sequence Synthetic polynucleotide
gctagcatgtgcctctctccggttaaaggcgcaaagctcatcctgatctttctgttcctaggagccgttcagtccaatgcagcgcgatgctctccgggggcgaaagtcaggtgcgctcgtggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatgagctcagcacccacaaagctagctggagcggaggcttgatagttaatttgacagattcaaagggtacttgcctttatgcagaatgggagatgaatttcacaataacatatgaaactacaaaccaaaccaataaaactataaccattgcagtacctgacaaggcgacacacgatggaagcagttgtggggatgaccggaatagtgccaaaataatgatacaatttggattcgctgtctcttgggctgtgaattttaccaaggaagcatctcattattcaattcatgacatcgtgctttcctacaacactagtgatagcacagtatttcctggtgctgtagctaaaggagttcatactgttaaaaatcctgagaatttcaaagttccattggatgtcatctttaagtgcaatagtgttttaacttacaacctgactcctgtcgttcagaaatattggggtattcacctgcaagcttttgtccaaaatggtacagtgagtaaaaatgaacaagtgtgtgaagaagaccaaactcccaccactgtggcacccatcattcacaccactgccccgtcgactacaactacactcactccaacttcaacacccactccaactccaactccaactccaaccgttggaaactacagcattagaaatggcaatactacctgtctgctggctaccatggggctgcagctgaacatcactgaggagaaggtgcctttcatttttaacatcaaccctgccacaaccaacttcaccggcagctgtcaacctcaaagtgctcaacttaggctgaacaacagccaaattaagtatcttgactttatctttgctgtgaaaaatgaaaaacggttctatctgaaggaagtgaatgtctacatgtatttggctaatggctcagctttcaacatttccaacaagaaccttagcttctgggatgcccctctgggaagttcttatatgtgcaacaaagagcaggtgctttctgtgtctagagcgtttcagatcaacacctttaacctaaaggtgcaaccttttaatgtgacaaaaggacagtattctacagctgaggaatgtgctgctgactctgacctcaactttcttattcctgttgcagtgggtgtggccttgggcttccttataattgctgtgtttatatcttacatgattggaagacggaaaagtcgtactggttatcagtctgtc

SEQ ID NO: 6 - DNA, 735 bp, Artificial Sequence
  Description of Artificial Sequence Synthetic polynucleotide
ggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatgagctcagcacccacaaagctagctgg

SEQ ID NO: 7 - protein, 6 aa, Artificial Sequence
  Description of Artificial Sequence Synthetic 6xHis tag
HHHHHH

(19) United States
(12) Patent Application Publication
(10) Pub. No.: US 2021/0130782 A1
(43) Pub. Date: May 6, 2021

(54) Engineered Exosomes to Detect and Deplete Pro-Tumorigenic Macrophages

(71) Applicant: Augusta University Research Institute, Inc., Augusta, GA (US)

(72) Inventors: Ali ARBAB, Augusta, GA (US); Mohammad Harun RASHID, Augusta, GA (US); Thaiz BORIN, Augusta, GA (US); Roxan ARA, Augusta, GA (US)

(73) Assignee: Augusta University Research Institute, Inc., Augusta, GA (US)

(21) Appl. No.: 17/083,124

(22) Filed: Oct. 28, 2020

Related U.S. Application Data
(60) Provisional application No. 62/926,775, filed on Oct. 28, 2019.

(51) Int. Cl. C12N 5/0786; A61K 35/15; A61P 35/00; A61P 35/04; G01N 33/574

(52) U.S. Cl. CPC C12N 5/0645; A61K 35/15; A61P 35/00; C12N 2501/2303; G01N 33/57492; C12N 2501/2304; A61P 35/04

(57) ABSTRACT

CD206-positive M2 macrophage-targeting exosomes and methods of use thereof are provided. One embodiment provides a CD206-positive M2 macrophage-targeting exosome expressing a CD206 binding peptide and an Fc portion of IgG2b. In some embodiments, the CD206 binding peptide is encoded by a nucleic acid sequence having 95%, 99%, or 100% sequence identity to SEQ ID NO:2 and the IgG2b is encoded by a sequence having 95%, 99%, or 100% sequence identity to SEQ ID NO:6.

### CROSS-REFERENCE TO RELATED APPLICATIONS

This application claims benefit of and priority to U.S. Provisional Patent Application No. 62/926,775 filed on Oct. 28, 2019, which is incorporated by reference in its entirety.

### STATEMENT REGARDING FEDERALLY SPONSORED RESEARCH

This invention was made with government support under RO1 CA160216 awarded by the National Institutes of Health. The government has certain rights in the invention.

### TECHNICAL FIELD OF THE INVENTION

Aspects of the invention are generally directed to compositions and methods of engineered exosomes for detecting and depleting pro-tumorigenic macrophages.

### BACKGROUND OF THE INVENTION

Exosomes have emerged as potential tools for a drug delivery system that can target specific tissues or cells. Recently, the therapeutic application of exosomes has shown promising results as novel therapeutic vehicles in cancer immunotherapy and suicide therapy, as well as delivery of RNA-interference and drugs (Yu et al., J. Immunol. 2007, 178, 6867-6875; El Andaloussi et al., Adv. Drug Deliv. Rev. 2013, 65, 391-397; El Andaloussi et al., Nat. Protocols 2012, 7, 2112-2126; Chaput et al., Cancer Immunol., Immunother.: CII 2004, 53, 234-239; Kurywchak et al., Genome Med. 2018, 10, 23 (2018)). Exosomes have clear advantages over synthetic nanoparticles like liposomes as a vehicle because of their improved biocompatibility, low toxicity and immunogenicity, permeability, stability in biological fluids, and ability to accumulate in the tumor with higher specificity (Mager et al, Nat. Rev. Drug Discov. 2013, 12, 347-357; Lener et al., J. Extracell. Vesicles 2015, 4, 30087; Jiang et al., Int. J. Pharm. 2017, 521, 167-175; Alvarez-Erviti et al., Nat. Biotechnol. 2011, 29, 341-345). Exosomes can be engineered to express targeting peptides or antibodies on their surface for precise targeted therapeutics delivery (Morishita et al., Biomaterials 2016, 111, 55-65; Stickney et al., Biochem. and Biophys. Res. Commun. 2016, 472, 53-59; Yim et al.; Nat Commun. 2016, 7, 12277).

Despite the exponential growth of chemotherapeutics and other targeted therapies for the treatment of cancer, there have been few successes for solid tumors. Thus, instead of focusing on the tumor cell alone, treatment strategies have been extended towards other cell types within the tumor microenvironment (TME). Increased infiltration of tumor associated macrophages (TAMs) correlates with tumor stage and poor survival. In addition to repolarization of macrophages, therapeutic depletion might be an attractive approach.

CD206-positive M2-macrophages are shown to have a pivotal role in the dissemination of breast cancer cells and prognosis (Williams et al., J. Clin. Oncol. 2018, 36, e24130-e24130; Linde et al., Nat. Commun. 2018, 9, 21). M2-macrophages participate in immune suppression, epithelial to mesenchymal transition, invasion, angiogenesis, tumor progression and subsequent metastasis foci formation. Investigators have utilized monoclonal antibody against CD206 or multi-mannose analog diagnostic imaging compounds that target the lectin domain of CD206 as imaging agents for detecting M2 macrophages in the TME or draining lymph nodes (Zhang et al.; Theranostics 2017, 7, 4276-4288; Scodeller et al., Scient. Rep. 2017, 7, 14655). In recent year, investigators have identified a peptide sequence CSPGAKVRC (SEQ ID NO:1) that binds specifically to CD206+ macrophages in the tumors and sentinel lymph nodes in different tumor models²². Generation of exosomes that uniquely bind to the receptor expressed by TAMs will enable the design of rational therapies that specifically target TAMs, ideally leaving normal macrophages unaffected.

Antibody-dependent cell-mediated cytotoxicity (ADCC) is a non-phagocytic mechanism by which most leucocytes (effector cells) can kill antibody-coated target cells in the absence of complement and without major histocompatibility complex (MEW) (van Dommelen et al., J. Controlled Release 2012, 161, 635-644). Targeted therapy utilizing monoclonal antibodies (mAbs) has instituted immunotherapy as a robust new tool to fight against cancer. As mAb therapy has revolutionized treatment of several diseases, ADCC has become more applicable in a clinical context. Clinical trials have demonstrated that many mAbs perform somewhat by eliciting ADCC (van der Meel et al., J. Controlled Release 2014, 195, 72-85). Antibodies serve as a bridge between Fc receptors (FcR) on the effector cell and the target antigen on the cell that is to be killed. There has not been any report of engineered targeted exosomes inducing ADCC. In the proposed model of engineered exosomes along with CD206 binding peptide, we conjugated Fc portion of the mouse IgG2b that could potentially be recognized by FcR on the effector cells and stimulate the ADCC events.

Therefore, it is an object of the invention to provide compositions of engineered exosomes for detecting and depleting pro-tumorigenic macrophages.

It is still another object of the invention to provide methods of engineered exosomes for detecting and depleting pro-tumorigenic macrophages.

### SUMMARY OF THE INVENTION

CD206-positive M2 macrophage-targeting exosomes and methods of use thereof are provided. One embodiment provides a CD206-positive M2 macrophage-targeting exosome expressing a CD206 binding peptide and an Fc portion of IgG2b. In some embodiments, the CD206 binding peptide is encoded by a nucleic acid sequence having 95%, 99%, or 100% sequence identity to SEQ ID NO:2 and the IgG2b is encoded by a sequence having 95%, 99%, or 100% sequence identity to SEQ ID NO:6.

Another embodiment provides a vector encoded by a nucleic acid sequence having 85%, 90%, 95%, or 100% to SEQ ID NO:5. The vector us useful for producing CD206-positive M2 macrophage-targeting exosomes. One embodiment provides a method for making CD206-positive M2 macrophage-targeting exosomes by transfecting macrophage with the vector, culturing the transfected macrophage in the presence of IL4 and IL-3, and harvesting the CD206-positive M2 macrophage-targeting exosomes. In some embodiments the macrophage are RAW264.7macrophage cells.

In some embodiments, the CD206-positive M2 macrophage-targeting exosomes are loaded with cargo. The cargo is selected from the group consisting of a detectable label, a chemotherapeutic agent, and a cytotoxic agent.

Another embodiment provides a pharmaceutical composition including the disclosed CD206-positive M2 macrophage-targeting exosomes and a pharmaceutically acceptable excipient.

Another embodiment provides a method of depleting M2 macrophage in a subject in need thereof by administering an effective amount of the a pharmaceutical composition including the disclosed CD206-positive M2 macrophage-targeting exosomes and a pharmaceutically acceptable excipient to the subject to deplete pro-tumorigenic macrophages including but not limited to M2 macrophage in the subject. In some embodiments the subject is a human.

In some embodiment, the subject has cancer, for example metastatic breast cancer.

Another embodiment provides a method for treating cancer in a subject in need thereof by administering an effective amount of the pharmaceutical composition including the disclosed CD206-positive M2 macrophage-targeting exosomes and a pharmaceutically acceptable excipient to the subject to deplete pro-tumorigenic macrophage including but not limited to M2 macrophage in the subject.

Another embodiment provides a method of reducing tumor burden in a subject in need thereof by administering an effective amount of the pharmaceutical composition including the disclosed CD206-positive M2 macrophage-targeting exosomes and a pharmaceutically acceptable excipient to the subject to reduce tumor burden in the subject.

Another embodiment provides a method for inducing antibody-dependent cell-mediated cytotoxicity in a subject in need thereof by administering an effective amount of the pharmaceutical composition including the disclosed CD206-positive M2 macrophage-targeting exosomes and a pharmaceutically acceptable excipient to the subject to induce antibody-dependent cell-mediated cytotoxicity in the subject.

Another embodiment provides a method for detecting cancer cells by contacting a biological sample with the CD206-positive M2 macrophage-targeting exosomes loaded with a detectable label and detecting the detectable label, wherein the detection of the label indicates the presence of cancer cells.

### BRIEF DESCRIPTION OF THE DRAWINGS

FIG. 1 represents generation of engineered exosomes expressing CD206-positive M2 macrophage-specific peptide along with Lamp2b.

FIG. 1a exhibits immunofluorescence staining of tumor, spleen and lungs sections from 4T1 tumor-bearing mice showing co-localization of Rhodamine red-labeled targeting peptide (injected i.v.) and FITC labeled CD206-positive M2-macrophages. Nuclei were visualized by DAPI staining (blue).

FIG. 1b is a schematic representation of the modified Lamp2b protein containing CD206 positive M2 macrophage-targeting peptide sequence following signal peptide, and a 6×HIS tag at the C terminus. Luciferase was used as a reporter gene.

FIG. 1c is a schematic diagram showing generation of CD206+M2-macrophage targeting engineered exosomes for diagnostic and therapeutic purpose.

FIG. 1d represents in vitro study showing luciferase activity of transfected HEK293 cells.

FIG. 1e is agarose gel electrophoresis showing confirmation of targeting peptide sequence insert in transfected HEK293 cells.

Figure if is a Western blot image showing anti-His tag antibody positivity in engineered exosomal protein content.

FIG. 1g and FIG. 1h showing size distribution by nanoparticle tracking assay (NTA) of the HEK293 exosomes and engineered exosomes, respectively. Quantitative data are expressed in mean±SEM.

FIG. 1i illustrates a transmission electron microscopy image for engineered exosomes, (Scale bar depicts 200 nm) showing characteristic round morphology and size without any deformity.

FIG. 2 represents targeting efficiency and specificity of CD206-positive M2 macrophage-specific exosomes.

FIG. 2a exhibits immunofluorescence staining showing targeting potential of DiI-labeled (red) engineered exosomes. RAW264.7 mouse macrophages were differentiated to CD206-positive (FITC) cells by treating with interleukin-4 and interleukin-13. Nuclei were visualized by DAPI staining (blue).

FIG. 2b exhibits immunofluorescence staining of mouse embryonic fibroblasts (MEFs) and RAW264.7 cells treated with or without anti-CD206 peptide, co-cultured with DiI-labeled (red) engineered exosomes. MEFs were negative for CD206 (FITC) staining and did not take up the exosomes. Engineered exosomes bound to the CD206+ RAW264.7 cells that was prevented by anti-CD206 peptide treatment.

FIG. 2c exhibits immunofluorescence staining of tumor, spleen and lungs sections from 4T1 tumor-bearing mice showing co-localization of rhodamine red-labeled targeting exosomes (injected i.v.) and FITC labeled CD206-positive M2-macrophages. Nuclei were visualized by DAPI staining (blue).

FIG. 2d exhibits stitched images for extended view of splenic section showing engineered exosomes were not taken up by T-lymphocytes and B-lymphocytes in splenic white pulp (white arrows).

FIG. 3 represents detection and quantification of biodistribution of ¹¹¹In-oxine-labeled exosomes targeting CD206-positive M2 macrophages.

FIG. 3a shows a major proportion of the free ¹¹¹In-oxine measured in the bottom to the top half of the thin layer paper chromatography (TLPC) paper, confirming the efficacy of the eluent.

FIG. 3b shows binding of ¹¹¹In-oxine to engineered exosomes was validated as shown by a lower percentage of ¹¹¹In-oxine (free, dissociated) measured in the top of the paper, compared to the amount remaining in the bottom, which represented the ¹¹¹In-oxine-labeled exosomes.

FIG. 3c shows serum stability of ¹¹¹In-oxine bound engineered exosomes was higher compared with the small amount of free ¹¹¹In-oxine disengaged from the bound exosomes.

FIG. 3d illustrates in vivo SPECT/CT images (coronal view) after 3 hrs of intravenous injection showed significant accumulation of M2-targeting exo in tumor, lung, spleen, lymph node and bones. ¹¹¹In-oxine-labeled non-targeting exosomes (HEK293 exo) and CD206-positive M2-macrophage targeting exosomes (M2-targeting exo) were injected into the 4T1 tumor-bearing mice. One group was treated with Clophosome® to deplete macrophages. Yellow and green arrows denote lymph node and bone metastasis, respectively.

FIG. 3e illustrates 3D surface images showing M2-targeting exo are profoundly distributed in both lung and tumor area compared to the group injected with HEK293 exo and pre-treated with Clophosome®. Yellow arrow indicates the tumor center.

FIG. 3f shows quantification of in vivo radioactivity in lungs, spleen and tumor.

FIG. 3g shows ex vivo radioactivity quantification in lungs, spleen and tumor. Quantitative data are expressed in mean±SEM. *P<0.05, **P<0.01, ***P<0.001, ****P<0.0001. n=3.

FIG. 4 represents generation of CD206-positive M2 macrophage-targeting therapeutic exosomes to induce antibody-dependent cell-mediated cytotoxicity.

FIG. 4a illustrates schematic diagram showing the proposed mechanism of engineered exosome-based antibody-dependent cellular cytotoxicity.

FIG. 4b illustrates schematic representation of the plasmid construct containing modified Lamp2b protein with CD206-targeting sequence conjugated with Fc segment of mouse IgG2b.

FIG. 4c demonstrates confirmation of luciferase activity by transfected HEK293 cells.

FIG. 4d shows flow cytometry analysis for validating the expression of Fc segment of mouse IgG2b on the surface of engineered exosomes. Three different engineered exosome samples were used for the flow cytometry.

FIG. 4e shows concentration and size distribution of the engineered therapeutic exosomes by nanoparticle tracking assay (NTA).

FIG. 4f shows mean diameter of engineered exosomes was significantly larger than non-engineered exosomes

FIG. 4g illustrates transmission electron microscopy image for engineered therapeutic exosomes, (Scale bar depicts 100 nm) showing distinctive round morphology and size without any distortion.

FIG. 4h shows flow-cytometry analysis of exosomal markers CD9 and CD63 for the engineered therapeutic exosomes. Three different engineered exosome samples were used for the flow cytometry.

FIG. 5 represents therapeutic efficiency and specificity of engineered therapeutic exosomes in depleting M2-macrophages both in vitro and in vivo.

FIG. 5a illustrates CFSE-labeled (green) RAW264.7 mouse macrophages were co-cultured with non-therapeutic CD206-positive cell-targeting exosomes (LAMP-206 exo) or CD206-positive cell-targeting therapeutic exosomes (LAMP-206-IgG2b exo), and without treatment (control) for 48 hours in presence of splenic immune cells from normal mice. Fluorescence microscopic images showed decrease in cell number and increased floating dead cells in LAMP-206-IgG2b exo group compared to other groups.

FIG. 5b shows measured fluorescence intensity of the above-mentioned conditions showed significant decrease in LAMP-206-IgG2b exo group compared to other groups.

FIG. 5c and FIG. 5d exhibit normal Balb/c mice were treated with one, two or three doses of engineered therapeutic exosomes expressing Fc portion of mouse IgG2b. Flow-cytometry analysis of splenic cells showing dose-dependent decline of F4/80 and CD206-positive M2-macrophage population.

FIG. 5e and FIG. 5f illustrate flow-cytometry analysis of splenic cells showing no significant change in both CD4 and CD8-positive T-cell population after treating the mice with different doses of therapeutic exosomes. Quantitative data are expressed in mean±SEM. *P<0.05, **P<0.01, ***P<0.001, ****P<0.0001. n=5.

FIG. 6 represents treatment of 4T1 tumor-bearing animals with therapeutic engineered exosomes prevent tumor growth and metastasis, and improve survival by depleting M2-macrophages.

FIG. 6a and FIG. 6b illustrate reconstructed and co-registered in vivo SPECT/CT images (coronal view) and quantification of subcutaneous syngeneic tumor-bearing animals (on the flank) injected with the 99mTc-labeled precision peptide after three hours. Group treated with therapeutic exosomes showed lesser level of radioactivity in tumor (yellow arrow) and spleen compared to untreated control group. Quantitative data are expressed in mean±SEM, *P<0.05. n=3.

FIG. 6c displays optical images of 4T1 tumor-bearing animals treated with engineered therapeutic exosomes (lower panel) or without treatment (control), showing decreased tumor growth in treated animals compared to control group. Metastatic foci in control group was detected (yellow arrows) as early as fourth week, whereas no metastasis was detected in treated animals after 6 weeks.

FIG. 6d illustrates quantification of optical density of the tumor area also showed decreased tumor growth in treated group compared to control group. Quantitative data are expressed in mean±SEM. n=3.

FIG. 6e shows Kaplan-Meier plot showing prolonged survival of the mice treated with therapeutic engineered exosomes.

FIG. 7 is a schematic of a representative plasmid used to produce CD206-positive M2 macrophage-targeting exosomes.

### DETAILED DESCRIPTION OF THE INVENTION

### I. Definitions

The following terms are intended to have the meanings presented therewith below and are useful in understanding the description and intended scope of the present invention.

The articles “a” and “an” may be used herein to refer to one or to more than one (i.e., at least one) of the grammatical objects of the article. By way of example “an analogue” means one analogue or more than one analogue.

As used herein, the term “pharmaceutical composition” means a mixture comprising a pharmaceutically acceptable active ingredient, in combination with suitable pharmaceutically acceptable excipients.

As used herein, the term “pharmaceutical formulation” means a composition in which different chemical substances, including the active drug, are combined to produce a final medicinal product. Examples of formulation include enteral formulations (tablets, capsules), parenteral formulations (liquids, lyophilized powders), or topical formulations (cutaneous, inhalable).

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable vehicle” refers to a diluent, adjuvant, excipient or carrier with which a compound of the invention is administered.

The term “Subject” includes mammals such as humans. The terms “human”, “patient” and “subject” are used interchangeably herein.

“Effective amount” means the amount of a compound of the invention that, when administered to a subject for treating a disease, is sufficient to effect such treatment for the disease. The ‘effective amount’ can vary depending on the compound, the disease and its severity, and the age, weight, etc., of the subject to be treated.

“Preventing” or “prevention” refers to a reduction in risk of acquiring or developing a disease or disorder (i.e., causing at least one of the clinical symptoms of the disease not to develop in a subject that may be exposed to a disease-causing agent, or predisposed to the disease in advance of disease onset).

The term “prophylaxis” is related to “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Treating” or “treatment” of any disease or disorder refers, in one embodiment, to ameliorating the disease or disorder (i.e., arresting the disease or reducing the manifestation, extent or severity of at least one of the clinical symptoms thereof). In another embodiment “reating” or “treatment” refers to ameliorating at least one physical parameter, which may not be discernible by the subject. In yet another embodiment, “treating” or “treatment′” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

### II. CD206-Positive M2 Macrophage-Targeting Exosome Compositions and Methods of Use

In recent years, several pioneers have explored the possibility of using exosomes as drug delivery vehicles. Owing to their defined size and natural function, exosomes appear ideal candidates for theranostic nanomedicine application. When compared to the administration of free drugs or therapeutics, exosomes have certain advantages such as improved stability, solubility, and in vivo pharmacokinetics. Exosomes can potentially increase circulation time, preserve drug therapeutic activity, increase drug concentration in the target tissue or cell to augment ther¬apeutic efficacy, while simultaneously reducing exposure of healthy tissues to reduce toxicity. Since they are nanosized and carry cell surface molecules, exosomes can cross various biological barriers, that might not be possible with free drugs or targeting agents.

One of the concerning factors for determining in vivo distribution in tumor model was enhanced permeability and retention (EPR) effect by which nanoparticles tend to concentrate in tumor tissue much more than they do in normal tissues. Although, only a fraction (0.7% median) of the total administered nanoparticle dose is usually able to reach a solid tumor, which might give false positive signals of exosome distribution. Surprisingly, we did not observe any retention of radioactivity for free ¹¹¹In-oxine, and non-targeted or non-cancerous exosomes (HEK293 exo). This implies that our demonstration of exosome biodistribution and targeted therapy is not an EPR effect, rather the exosomes were directed toward target organs by over-expressed precision peptide on their surface. Many mechanisms have been implemented to boost the antitumor activities of therapeutic antibodies, including extended half-life, blockade of signaling pathways, activation of apoptosis and effector-cell-mediated cytotoxicity. Here we propose to target Fc gamma-receptor (FcR) based platform to deplete of M2 macrophages. The direct effector functions that result from FcR triggering are phagocytosis, ADCC, and induction of inflammation; also, FcR-mediated processes provide immune-regulation and immunomodulation that augment T-cell immunity and fine-tune immune responses against antigens. With respect to IgG2b, part of the most potent IgG subclasses can bind specifically into FcRIII (KD=1.55×10⁻⁶) and IV (KD=5.9×10⁻⁸) to activate FcRs.^{[35,36]} Peptibodies containing myeloid-derived suppressor cells (MDSC)-specific peptide fused with Fc portion of IgG2b was able to deplete MDSCs in vivo and retard tumor growth of a lymphoma mouse model without affecting proinflammatory immune cells types, such as dendritic cells.^{[37]} This plasticity of effector and immune-regulatory functions offers unique opportunities to apply FcR-based platforms and immunotherapeutic regimens for vaccine delivery and drug targeting against infectious and non-infectious diseases.

Investigators have used tumor cells, dendritic cells (DCs), mes-enchymal stem cells (MSCs), MDSCs, endothelial progenitor cells (EPCs), neural stem cells (NSCs), and other cell types to generate engineered and non-engineered exosomes for both imaging and therapeutic purpose. We have also used tumor cells, MDSCs, EPCs, and NSCs derived exosomes in our previous and ongoing studies. Tumor cell-derived exosomes carry antigens and elicit immunogenic reaction, therefore, these ex-osomes have been used in studies for tumor vaccination. On the other hand, both MSCs and MDSCs derived exosomes have shown to be immune suppressive. EPC-derived ex-osomes may enhance neovascularization in the tumors. Therefore, using these cells to generate engineered exosomes to carry CD206 targeting peptide may initiate unwanted effect of immune activation, immune suppression, or neovasculariza-tion. Moreover, in vitro growth of MSCs, NSCs, and EPCs may be limited due to cell passage number. Ideal cell to generate engineered exosomes should have the following criteria: 1) Non-immunogenic, 2) unlimited cell passage capacity without changing their characteristics, 3) abundant production of exosomes both in normal and strenuous conditions, 4) cells that can easily be genetically modified. HEK 293 cell is ideal for the production of engineered exosomes. These cells have been extensively used by the biotechnology industry to produce FDA (food and Drug Administration) approved therapeutic proteins and viruses for gene therapies. Exosomes derived from these cells show no immune activation or suppression following long-term injections in animal models. We used HEK293 cells to generate our engineered exosomes to carry precision peptide to target CD206+M2 macrophages.

The data provided in the Examples shows that exosomes targeting M2 macrophages are utilized effectively to diagnose, monitor, and prevent tumor growth and metastasis for better survival.

A. Compositions

CD206-positive M2 macrophage-targeting exosomes and methods of use thereof are provided. One embodiment provides a CD206-positive M2 macrophage-targeting exosome expressing a CD206 binding peptide and an Fc portion of IgG2b. In some embodiments, the CD206 binding peptide is encoded by a nucleic acid sequence having 95%, 99%, or 100% sequence identity to SEQ ID NO:2 and the IgG2b is encoded by a sequence having 95%, 99%, or 100% sequence identity to SEQ ID NO:6.

Another embodiment provides a vector encoded by a nucleic acid sequence having 85%, 90%, 95%, or 100% to SEQ ID NO:5. The vector us useful for producing CD206-positive M2 macrophage-targeting exosomes. One embodiment provides a method for making CD206-positive M2 macrophage-targeting exosomes by transfecting macrophage with the vector, culturing the transfected macrophage in the presence of IL4 and IL-3, and harvesting the CD206-positive M2 macrophage-targeting exosomes. In some embodiments the macrophage are RAW264.7macrophage cells.

In some embodiments, the CD206-positive M2 macrophage-targeting exosomes are loaded with cargo. The cargo is selected from the group consisting of a detectable label, a chemotherapeutic agent, and a cytotoxic agent.

Another embodiment provides a pharmaceutical composition including the disclosed CD206-positive M2 macrophage-targeting exosomes and a pharmaceutically acceptable excipient.

B. Methods of Use

Another embodiment provides a method of depleting M2 macrophage in a subject in need thereof by administering an effective amount of the a pharmaceutical composition including the disclosed CD206-positive M2 macrophage-targeting exosomes and a pharmaceutically acceptable excipient to the subject to deplete pro-tumorigenic macrophages including but not limited to M2 macrophage in the subject. In some embodiments the subject is a human.

In some embodiment, the subject has cancer, for example metastatic breast cancer.

Representative cancer that can be inhibited or treated by the compound of formula I or pharmaceutical composition thereof includes, but are not limited to, squamous cell carcinoma, small-cell lung cancer, non-small cell lung cancer (NSCLC), lung adenocarcinoma, squamous cell lung cancer, peritoneum cancer, hepatocellular cancer, stomach cancer, gastrointestinal cancer, esophageal cancer, pancreatic cancer, glioblastoma, cervical cancer, ovarian cancer, liver cancer, bladder cancer, breast cancer, colon cancer, rectal cancer, colorectal cancer, endometrial cancer, uterine cancer, salivary gland carcinoma, renal cancer, prostate cancer, vulval cancer, thyroid cancer, hepatocellular carcinoma (HCC), anal carcinoma, penile carcinoma, or head and neck cancer.

Another embodiment provides a method for treating cancer in a subject in need thereof by administering an effective amount of the pharmaceutical composition including the disclosed CD206-positive M2 macrophage-targeting exosomes and a pharmaceutically acceptable excipient to the subject to deplete pro-tumorigenic macrophage including but not limited to M2 macrophage in the subject.

Another embodiment provides a method of reducing tumor burden in a subject in need thereof by administering an effective amount of the pharmaceutical composition including the disclosed CD206-positive M2 macrophage-targeting exosomes and a pharmaceutically acceptable excipient to the subject to reduce tumor burden in the subject.

Another embodiment provides a method for inducing antibody-dependent cell-mediated cytotoxicity in a subject in need thereof by administering an effective amount of the pharmaceutical composition including the disclosed CD206-positive M2 macrophage-targeting exosomes and a pharmaceutically acceptable excipient to the subject to induce antibody-dependent cell-mediated cytotoxicity in the subject.

Another embodiment provides a method for detecting cancer cells by contacting a biological sample with the CD206-positive M2 macrophage-targeting exosomes loaded with a detectable label and detecting the detectable label, wherein the detection of the label indicates the presence of cancer cells.

C. Combination Therapies

In some embodiments the CD206-positive M2 macrophage-targeting exosomes are administered in combination or alternation with a second therapeutic agent.

1. Chemotherapeutic Agents

CD206-positive M2 macrophage-targeting exosomes can be combined with or loaded with one or more chemotherapeutic agents and pro-apoptotic agents. Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

2. Other Immunomodulators

### PD-1 Antagonists

In some embodiments, CD206-positive M2 macrophage-targeting exosomes are co-administered with a PD-1 antagonist. Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, which are specifically incorporated by reference herein in their entities, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U. S. A, 105:10275-10276 (2008)). Therefore, proteins, antibodies or small molecules that prevent co-ligation of PD-1 and TCR on the T cell membrane are also useful PD-1 antagonists.

In some embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

Other PD-1 antagonists contemplated by the methods of this invention include antibodies that bind to PD-1 or ligands of PD-1, and other antibodies.

Suitable anti-PD-1 antibodies include, but are not limited to, those described in the following U.S. Pat. Nos. 7,332,582, 7,488,802, 7,521,051, 7,524,498, 7,563,869, 7,981,416, 8,088,905, 8,287,856, 8,580,247, 8,728,474, 8,779,105, 9,067,999, 9,073,994, 9,084,776, 9,205,148, 9,358,289, 9,387,247, 9,492,539, 9,492,540—all of which are incorporated by reference in their entireties. See also Berger et al., Clin. Cancer Res., 14:30443051 (2008).

Exemplary anti-B7-H1 (also referred to as anti-PD-L1) antibodies include, but are not limited to, those described in the following U.S. Pat. Nos. 8,383,796, 9,102,725, 9,273,135, 9,393,301, and 9,580,507 all of which are specifically incorporated by reference herein in their entirety.

For anti-B7-DC (also referred to as anti-PD-L2) antibodies see U.S. Pat. Nos. 7,411,051, 7,052,694, 7,390,888, 8,188,238, and 9,255,147 all of which are specifically incorporated by reference herein in their entirety.

Other exemplary PD-1 receptor antagonists include, but are not limited to B7-DC polypeptides, including homologs and variants of these, as well as active fragments of any of the foregoing, and fusion proteins that incorporate any of these. In some embodiments, the fusion protein includes the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, for example from mouse or primate, such as a human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

### CTLA4 Antagonists

In some embodiments, the CD206-positive M2 macrophage-targeting exosomes are administered in combination or alternation with molecule an antagonist of CTLA4, for example an antagonistic anti-CTLA4 antibody. An example of an anti-CTLA4 antibody contemplated for use in the methods of the invention includes an antibody as described in PCT/US2006/043690 (Fischkoff et al., WO/2007/056539).

Dosages for anti-PD-1, anti-B7-H1, and anti-CTLA4 antibody, are known in the art and can be in the range of, for example, 0.1 to 100 mg/kg, or with shorter ranges of 1 to 50 mg/kg, or 10 to 20 mg/kg. An appropriate dose for a human subject can be between 5 and 15 mg/kg, with 10 mg/kg of antibody (for example, human anti-PD-1 antibody) being a specific embodiment.

Specific examples of an anti-CTLA4 antibody useful in the methods of the invention are Ipilimumab, a human anti-CTLA4 antibody, administered at a dose of, for example, about 10 mg/kg, and Tremelimumab a human anti-CTLA4 antibody, administered at a dose of, for example, about 15 mg/kg. See also Sammartino, et al., Clinical Kidney Journal, 3(2):135-137 (2010), published online December 2009.

In other embodiments, the antagonist is a small molecule. A series of small organic compounds have been shown to bind to the B7-1 ligand to prevent binding to CTLA4 (see Erbe et al., J. Biol. Chem., 277:7363-7368 (2002). Such small organics could be administered alone or together with an anti-CTLA4 antibody to reduce inhibitory signal transduction of T cells.

### Potentiating Agents

In some embodiments, additional therapeutic agents include a potentiating agent. The potentiating agent acts to increase efficacy the immune response up-regulator, possibly by more than one mechanism, although the precise mechanism of action is not essential to the broad practice of the present invention.

In some embodiments, the potentiating agent is cyclophosphamide. Cyclophosphamide (CTX, Cytoxan®, or Neosar®) is an oxazahosphorine drug and analogs include ifosfamide (IFO, Ifex), perfosfamide, trophosphamide (trofosfamide; Ixoten), and pharmaceutically acceptable salts, solvates, prodrugs and metabolites thereof (US patent application 20070202077 which is incorporated in its entirety). Ifosfamide (MITOXANA®) is a structural analog of cyclophosphamide and its mechanism of action is considered to be identical or substantially similar to that of cyclophosphamide. Perfosfamide (4-hydroperoxycyclophosphamide) and trophosphamide are also alkylating agents, which are structurally related to cyclophosphamide. For example, perfosfamide alkylates DNA, thereby inhibiting DNA replication and RNA and protein synthesis. New oxazaphosphorines derivatives have been designed and evaluated with an attempt to improve the selectivity and response with reduced host toxicity (Liang J, Huang M, Duan W, Yu X Q, Zhou S. Design of new oxazaphosphorine anticancer drugs. Curr Pharm Des. 2007; 13(9):963-78. Review). These include mafosfamide (NSC 345842), glufosfamide (D19575, beta-D-glucosylisophosphoramide mustard), S-(−)-bromofosfamide (CBM-11), NSC 612567 (aldophosphamide perhydrothiazine) and NSC 613060 (aldophosphamide thiazolidine). Mafosfamide is an oxazaphosphorine analog that is a chemically stable 4-thioethane sulfonic acid salt of 4-hydroxy-CPA. Glufosfamide is IFO derivative in which the isophosphoramide mustard, the alkylating metabolite of IFO, is glycosidically linked to a beta-D-glucose molecule. Additional cyclophosphamide analogs are described in U.S. Pat. No. 5,190,929 entitled “Cyclophosphamide analogs useful as anti-tumor agents” which is incorporated herein by reference in its entirety.

While CTX itself is nontoxic, some of its metabolites are cytotoxic alkylating agents that induce DNA crosslinking and, at higher doses, strand breaks. Many cells are resistant to CTX because they express high levels of the detoxifying enzyme aldehyde dehydrogenase (ALDH). CTX targets proliferating lymphocytes, as lymphocytes (but not hematopoietic stem cells) express only low levels of ALDH, and cycling cells are most sensitive to DNA alkylation agents.

Low doses of CTX (<200 mg/kg) can have immune stimulatory effects, including stimulation of anti-tumor immune responses in humans and mouse models of cancer (Brode & Cooke Crit Rev. Immunol. 28:109-126 (2008)). These low doses are sub-therapeutic and do not have a direct anti-tumor activity. In contrast, high doses of CTX inhibit the anti-tumor response. Several mechanisms may explain the role of CTX in potentiation of anti-tumor immune response: (a) depletion of CD4+CD25+ FoxP3+Treg (and specifically proliferating Treg, which may be especially suppressive), (b) depletion of B lymphocytes; (c) induction of nitric oxide (NO), resulting in suppression of tumor cell growth; (d) mobilization and expansion of CD11b+Gr−1+MDSC. These primary effects have numerous secondary effects; for example following Treg depletion macrophages produce more IFN-γ and less IL-10. CTX has also been shown to induce type I IFN expression and promote homeostatic proliferation of lymphocytes.

Treg depletion is most often cited as the mechanism by which CTX potentiates the anti-tumor immune response. This conclusion is based in part by the results of adoptive transfer experiments. In the AB1-HA tumor model, CTX treatment at Day 9 gives a 75% cure rate. Transfer of purified Treg at Day 12 almost completely inhibited the CTX response (van der Most et al. Cancer Immunol. Immunother. 58:1219-1228 (2009). A similar result was observed in the HHD2 tumor model: adoptive transfer of CD4+CD25+Treg after CTX pretreatment eliminated therapeutic response to vaccine (Taieb, J. J. Immunol. 176:2722-2729 (2006)).

Numerous human clinical trials have demonstrated that low dose CTX is a safe, well-tolerated, and effective agent for promoting anti-tumor immune responses (Bas, & Mastrangelo Cancer Immunol. Immunother. 47:1-12 (1998)).

The optimal dose for CTX to potentiate an anti-tumor immune response, is one that lowers overall T cell counts by lowering Treg levels below the normal range but is subtherapeutic (see Machiels et al. Cancer Res. 61:3689-3697 (2001)).

In human clinical trials where CTX has been used as an immunopotentiating agent, a dose of 300 mg/m2 has usually been used. For an average male (6 ft, 170 pound (78 kg) with a body surface area of 1.98 m2), 300 mg/m2 is 8 mg/kg, or 624 mg of total protein. In mouse models of cancer, efficacy has been seen at doses ranging from 15-150 mg/kg, which relates to 0.45-4.5 mg of total protein in a 30 g mouse (Machiels et al. Cancer Res. 61:3689-3697 (2001), Hengst et al Cancer Res. 41:2163-2167 (1981), Hengst Cancer Res. 40:2135-2141 (1980)).

For larger mammals, such as a primate, such as a human, patient, such mg/m2 doses may be used but unit doses administered over a finite time interval may also be used. Such unit doses may be administered on a daily basis for a finite time period, such as up to 3 days, or up to 5 days, or up to 7 days, or up to 10 days, or up to 15 days or up to 20 days or up to 25 days, are all specifically contemplated by the invention. The same regimen may be applied for the other potentiating agents recited herein.

In other embodiments, the potentiating agent is an agent that reduces activity and/or number of regulatory T lymphocytes (T-regs), such as Sunitinib (SUTENT®), anti-TGFβ or Imatinib (GLEEVAC®). The recited treatment regimen may also include administering an adjuvant.

Useful potentiating agents also include mitosis inhibitors, such as paclitaxol, aromatase inhibitors (e.g. Letrozole) and angiogenesis inhibitors (VEGF inhibitors e.g., Avastin, VEGF-Trap) (see, for example, Li et al., Clin Cancer Res. 2006 Nov. 15; 12(22):6808-16.), anthracyclines, oxaliplatin, doxorubicin, TLR4 antagonists, and IL-18 antagonists.

### III. Pharmaceutical Formulations

The CD206-positive M2 macrophage-targeting exosomes and mixtures thereof can be formulated into a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration. The compositions can be administered systemically.

The CD206-positive M2 macrophage-targeting exosomes can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g., as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et. al., (Media, Pa.: Williams and Wilkins, 1995) which provides information on carriers, materials, equipment and process for preparing tablets and capsules and delayed release dosage forms of tablets, capsules, and granules.

The CD206-positive M2 macrophage-targeting exosomes can be administered to a subject with or without the aid of a delivery vehicle. Appropriate delivery vehicles for the compounds are known in the art and can be selected to suit the particular active agent. For example, in some embodiments, the active agent(s) is/are incorporated into or encapsulated by, or bound to, a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

A. Formulations for Parenteral Administration

CD206-positive M2 macrophage-targeting exosomes and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

B. Extended Release Dosage Forms

The extended release formulations of CD206-positive M2 macrophage-targeting exosomes are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations of CD206-positive M2 macrophage-targeting exosomes can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

C. Delayed Release Dosage Forms

In some embodiments delayed release formulations of CD206-positive M2 macrophage-targeting exosomes are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUIDRAGIT®. L-100 (soluble at pH 6.0 and above), EUDRAGIT®. S (soluble at pH 7.0 and above, as a result of a higher degree of esterification), and EUIDRAGIT®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### Methods and Materials

### Cell Lines

4T1, a murine mammary carcinoma cell line from a BALB/cfC3H mouse, was originally obtained from the American Type Tissue Culture Collection (ATCC), and modified by Dr. Hasan Korkaya (Augusta University) to express the luciferase gene reporter. For cell cultures and propagation, both cells were grown in Roswell Park Memorial Institute 1640 medium (RPMI) (Thermo Scientific), supplemented with 10% fetal bovine serum (FBS) (Nalgene-GIBCO), 2 mM glutamine (GIBCO, Grand Island, N.Y., USA) and 100 U/mL penicillin and streptomycin (GIBCO, Grand Island, N.Y., USA) at 5% CO₂ at 37° C. in a humidified incubator. For the generation of exosomes, cells (5×10⁶ cells in T175 flask) were grown in RPMI-1640 media containing 10% exosome free FBS and incubated in a humidified incubator in hypoxic condition (1% oxygen) for 48 hours. Mouse Embryonic Fibroblast cell line (MEF) was obtained from Dr. Nahid Mivechi's laboratory and both cell lines and Human embryonic kidney 293 cell line (HEK293) was obtained from Dr. Satyanarayana Ande of Augusta University were grown in Dulbecco's Modified Eagle Medium (DMEM) (Corning, N.Y., USA) containing 10% exosome free FBS. HEK293 cells were transfected with lentivirus to develop engineered exoxomes. RAW264.7 mouse macrophage cell line was obtained from Dr. Gabor Csanyi in the vascular biology department at Augusta University and used for in vitro targeting and cytotoxicity assays. RAW264.7 were grown in DMEM media containing 10% FBS.

### Exosome Isolation

Exosomes were isolated from the culture supernatants of 4T1, HEK293 cells and transfected HEK293 cells. Briefly, 5×10⁶ cells were plated in 175 cm2 flasks and grown overnight with 10% FBS complete media in normoxia (20% oxygen). The media was removed and replenished with exosome-free complete media. Exosomes were depleted from the complete media by ultracentrifugation for 70 minutes at 100,000×g using an ultracentrifuge (Beckman Coulter) and SW28 swinging-bucket rotor. The cells were then grown for 48 hours under normoxic condition. The cell culture supernatant was centrifuged at 700×g for 15 minutes to get rid of cell debris. To isolate exosomes, we employed combination of two steps of size-based method by passing through 0.20 μm syringe filter and centrifugation with 100 k membrane tube at 3200×g for 30 minutes followed by a single step of ultracentrifugation at 100,000×g for 70 minutes (as described in our previous publication²⁶.

### Nanoparticle Tracking Analysis

Nanoparticle tracking analysis (NTA) was performed using ZetaView, a second-generation particle size instrument from Particle Metrix for individual exosome particle tracking as described previously²⁶. This is a high performance integrated instrument equipped with a cell channel, which is integrated into a ‘slide-in’ cassette and a 405-nm laser. Samples were diluted in 1×PBS between 1:100 and 1:2000 and injected in the sample chamber with sterile syringes (BD Discardit II, New Jersey, USA). All measurements were performed at 23° C. and pH 7.4. As measurement mode, we used 11 positions with 2 cycles, and for analysis parameter, we used maximum pixel 200 and minimum 5. ZetaView 8.02.31 software and Camera 0.703 μm/px were used for capturing and analyzing the data.

### Flow Cytometry

The common exosome markers, mouse-specific anti-CD9 FITC, and anti-CD63 APC antibody (Biolegend, San Diego, Calif., USA) were used to label exosomes at 4° C. for 30 minutes. Flow cytometry samples were acquired using Accuri C6 flow cytometer (BD Biosciences) with the threshold set at 10 and analyzed by BD Accuri C6 software. For the in vivo flow cytometric analysis, the fresh tissue collected was disseminated into single cells, filtered through a 70 μm cell strainer, and spun at 1,200 rpm for 15 minutes. The pellet was re-suspended in 1% BSA/PBS, and incubated with LEAF blocker in 100 μL volume for 15 minutes on ice to reduce non-specific staining. The single cells were then labeled to detect the macrophage and immune cell populations using fluorescence conjugated antibodies such as CD3, CD4, CD8, CD206, F4/80 and IgG2b. All antibodies were mouse specific and the samples were acquired using Accuri C6 flow cytometer (BD Biosciences).

### Tumor Model

4T1 cells expressing the luciferase gene were orthotopically implanted in syngeneic BALB/c (Jackson Laboratory, Main USA). All the mice were between 5-6 weeks of age and weighing 18-20 g. Animals were anesthetized using a mixture of Xylazine (20 mg/Kg) and Ketamine (100 mg/Kg) administered intraperitoneally. Hair was removed for the right half of the abdomen by using hair removal ointment, and then abdomen was cleaned by Povidone-iodine and alcohol. A small incision was made in the middle of the abdomen, and the skin was separated from the peritoneum using blunt forceps. Separated skin was pulled to the right side to expose the mammary fat pad and 50,000 4T1 cells in 504, Matrigel (Corning, N.Y., USA) were injected. Tumor growth was monitored every week. In vivo, optical images were obtained every week to keep track of primary tumor and metastasis development by injecting 100 μL of luciferin (dose 150 mg/kg) intraperitoneally followed by the acquisition of bioluminescence signal by spectral AmiX optical imaging system (Spectral instruments imaging, Inc. Tucson, Ariz.). The photon intensity/mm/sec was determined by Aura imaging software by Spectral Instruments Imaging, LLC (version 2.2.1.1). The animals were anesthetized using an isoflurane vaporizer chamber (2.5% Iso: 2±3 L/min 02) and maintained under anesthesia (2% with oxygen) during the procedure.

### Radiolabeling of Exosomes Using Indium-111 (¹¹¹In)

Exosomes were labeled with In-111-oxine using our optimized method of labeling²³. In brief, exosomes (fresh or thawed) were washed with normal saline, reconstituted at 12 billion exosomes/ml, incubated with 1 mCi of In-111-oxine in normal saline for 30 minutes at room temperature. Then free from bound In-111 will be separated using Amicon ultra centrifugal filters with a cut off value of 100 kDa for 30 minutes at 3200×g at 20° C. Serum challenge studies were used to determine any dissociation over 24 hours, which was determined by thin-layered paper chromatography (TLPC).

### Thin Layer Paper Chromatography for Radiolabeling Efficacy and Stability

3 MM Whatman® cellulose chromatography paper was cut into 1×8 cm small pieces. The bottom spotted point was made by 54, of each sample followed by submerging the bottom part of each piece (fluid level remained below the spotted point) into the eluent consisting of 100% methanol and 2M Sodium acetate solution (1:1). Then the pieces were allowed to remain upright until the eluent reaches the top part. The pieces were cut into the top and bottom halves and were subsequently put in the glass tubes for the measurement of emitted gamma activity by Perkin-Elmer Packard Cobra II Auto-Gamma. Total radioactivity was calculated by combining the activity from top and bottom halves. To determine the percent dissociation of bound ¹¹¹In-oxine from exosomes, labeled exosomes were challenged with serum at 37° C. up to 24 hrs or 48 hrs. At different time points, free ¹¹¹In-oxine, and serum challenged labeled exosomes were tested using thin layer paper chromatography as described above to determine the percent of bound vs. free ¹¹¹In-oxine.

### In Vivo SPECT/CT Imaging of ¹¹¹In-Oxine-Labeled Exosomes

After the intravenous injection of 350±50 μCi of ¹¹¹In-oxine-labeled exosomes in 100 into the tail vein of the mice, whole body SPECT images were acquired using our previously published protocol with a dedicated 4-headed NanoScan, high-sensitivity microSPECT/CT 4R (Mediso, Boston, Mass., USA) fitted with high-resolution multi-pinhole (total 100) collimators. The microSPECT has a wide range of energy capabilities from 20 to 600 keV, with a spatial resolution of 275 μm. The images were obtained using 60 projection images with 60 seconds/projection, with a medium field of view. Attenuation was corrected using concurrent computed tomographic (CT) images, and then the images were reconstructed with low iteration and low filtered back-projection. The image acquisitions were commenced 3 hours after the injection of ¹¹¹In-oxine-labeled exosomes. During the whole procedure, the animals were anesthetized and maintained using a combination of 1.5% isoflurane and 1 L/min medical oxygen flow and their body was immobilized in an imaging chamber to restrain movements. Throughout the scanning their body temperature was maintained at 37° C. and breathing was monitored.

### Quantitative Analysis of Radioactivity in Individual Organ

Reconstructed analyze formatted file was used in ImageJ (Wayne Rasband, National Institutes of Health, USA) version 1.51a for both CT and SPECT analysis. The primary tumor, a metastatic site in the lungs and other organs were identified by orthogonal, dorsal and ventral views from the resliced stack images. Z stack images were created from the CT and SPECT of the individual organ for depth and anatomical accuracy of the organ. Total radioactivity was determined by the sum of the values of the pixels (RawIntDen) in the selected region of interest (ROIs) around the organs. The activity in the individual organ was expressed in percent of activity in the whole body (total radioactivity dose).

### Ex Vivo Quantification of Gamma Activity of Individual Organ

After the final scan, animals were euthanized, and their organs were harvested and weighed. Emitted gamma radiation from each organ was measured by Perkin-Elmer Packard Cobra II Auto-Gamma after transferring them into the individual glass tube.

### Determination of Specificity of Precision Peptide In Vitro and In Vivo

Biotinylated precision peptide (Biotin-CSPGAKVRC) (SEQ ID NO:1) was custom synthesized by a commercial vendor (GeneScript, Piscataway, N.J.) using standard peptide synthesis and biotin was attached to the N-terminus. For both in vitro and in vivo studies, biotinylated peptide was labeled with rhodamine using rhodamine-tagged streptavidin utilizing standard protocol for labeling supplied by the vendor (ThermoFisher Scientific). Rhodamine-labeled peptide was used in in vitro studies to determine the specific uptake to CD206 sites on RAW 264.7 cells with or without blocking CD206 receptor using a CD206 blocking peptide (Cat #MB S823969, mybiosource.com). All cells were pre-incubated with anti-CD44 antibody to block non-specific phagocytosis. All cells were stained for CD206 (fluorescein, FITC) and counter stained with DAPI.

For in vivo specificity, rhodamine labeled peptide (red) was injected intravenously (IV) in metastatic syngeneic murine breast cancer (4T1) bearing Balb/C mice. Three hours after IV administration, all animals were euthanized, and lungs, spleen and tumors were collected for immunohistochemical analysis. Frozen sections from the collected tissues were stained for CD206 (fluorescein, FITC) and counter stained with DAPI.

Labeling of Conjugated-Precision Peptide with Tc99m:

Hydrazine Nicotinamide (HYNIC)-conjugated M2-targeting precision peptide was custom synthesized by a commercial vendor (GeneScript, Piscataway, N.J.) using standard peptide synthesis. Then, 250 μg of HYNIC-M2-targeting conjugated peptide was radio labeled with 99m-Tc-pertechnetate in the presence of a solution containing tricine (14.4 mg/mL—Acros organics) and stannous chloride (0.5 mg/mL—Acros organics) in oxygen free condition (air was purged by N2). Following this step, we centrifuged the mixture to remove the unconjugated peptide using 1K centrifugal filter at 3200×g for 15 min. The amount of radiolabeled peptide was detected using a dose calibrator (CRC-25R—Capintec, Inc.). A dose of approximately 300 μCi of radiolabeled peptide was injected per animal.

### Construction for Overexpressing CD206+M2-Macrophage Targeting Peptide and Fc Portion of Mouse IgG2b on the Exosome Surface:

We had two different lentiviral vector constructs made by 3rd party vendor (VectorBuilder Inc, TX, USA), which were used to generate engineered exosomes in HEK293 cells. CD206+M2-macrophage targeting peptide and Fc portion of mouse IgG2b along with mouse LAMP2b protein were custom designed and inserted into third-generation lentivirus vector (eBiosciences). QIAquick Gel Extraction Kit (Qiagen, Valencia, Calif., USA) and Plasmid Midi Kit (Qiagen, Valencia, Calif., USA) were used to extracting the plasmid DNA.

### Biogenesis of Engineered Exosomes Expressing Precision Peptide and Fusion Protein

For the lentiviral production, we seeded 1×10⁶ HEK293TN cells in a 100 mm culture dish. At 70-75% of confluency, after removing the old media, we supplemented the cells with lentivirus producing plasmids and our targeting cloning plasmid in the presence of Opti-mem and Lipofectamine2000. After 24 hours, we collected the culture supernatants containing virus particles followed by centrifugation and filtration through 0.45 μm PVDF membrane to get rid of the cell debris. For the transfection using lentivector, we seeded 500,000 HEK293 cells in a 100 mm culture dish. At 70-75% of confluency, after removing the old media, we supplemented the cells with transfection cocktail containing regular media, lentivirus, and polybrene. The cells were expanded and subsequently selected with 300 μg/mL neomycin for 4 weeks. The transfection of selected cells was confirmed by luciferase activity of the cells following the addition of luciferin. After collecting the supernatant from 6×10⁶ transfected HEK293 cell cultures incubated for 48 hours in a T175 flask with exosomes free media, the supernatant was centrifuged at 700×g for 15 minutes to remove cell debris. Then it was filtered through a 0.20 μm PVDF (low protein attachment) membrane and centrifuged using Amicon ultra centrifugal filters with a cut off value of 100 kDa for 30 minutes at 3200×g followed by a final washing step with ultracentrifugation at 100,000×g for 70 minutes.

Labeling of Exosomes with DiI

DiI-labeled exosomes were used to demonstrate targeting efficiency of the engineered exosomes both in vitro and in vivo. Following isolation, exosomes were re-suspended in 1 mL of DiI working solution (final concentration 5 μM/mL in PBS). After 30 minutes of incubation at 37° C., free DiI was removed by two centrifugation wash steps with PBS using 100 k membrane tubes.

### Immunofluorescent Staining of Adherent Cell Cultures

18−18-1 glass coverslips were soaked in 100% ethanol for sterilization followed by washing in PBS and then each of them was transferred to each well of 6 well-plates. 300,000 RAW264.7 cells were seeded and incubated overnight. Then the adherent cells were treated with DiI-labeled exosomes (204, containing approximately 3×10⁸ exosomes) and incubated for 4-6 hours. After that, media with exosomes was removed and the cells were rinsed twice with PBS. Cells were fixed with 3% paraformaldehyde for 15 minutes followed by washing with PBS. Cells were covered with blocking solution and incubated for 20-30 minutes at room temperature. Blocking solution was gently flicked away and appropriate antibody (Alexa 488 anti-mouse CD206 antibody) diluted in blocking solution (1:100) was added. After 2 hours of incubation the antibody was removed and the cells were washed with PBS followed by counter staining with DAPI for nuclear stain. After final wash step, the coverslips were transferred for mounting on slides using ProLong™ Gold Antifade mounting media (Invitrogen™)

### Determination of Specificity of Engineered Exosomes In Vitro and In Vivo

In vitro studies: Raw264.7 (CD206+ cells) and mouse embryonic fibroblast (MEF, CD206-cells) were used as model cells for in vitro studies of CD206 specificity for engineered exosomes. The anti-CD44 antibody was used before adding the exosomes to block the non-specific uptake of added exosomes by the process of phagocytosis. Both Raw264.7 and MEF cells, grown in small tissue culture petri-dish, were treated with anti-CD44 antibody to block phagocytosis, and then these cells were incubated with fluorescent dye DiI labeled engineered and control exosomes collected from HEK293 cells with or without CD206 blocking peptide (Cat #MBS823969, mybiosource.com). CD206 blocking peptide was used to determine the specificity of the engineered exosomes expressing precision CD206 targeting peptide to target CD206 sites. Cells were stained with an anti-CD206 antibody plus FITC tagged secondary antibody. High-resolution fluorescent microscopy images were obtained.

In vivo studies using DiI labeled exosomes For in vivo specificity studies, we used Balb/c mice bearing 4T1 tumors, which were treated with either vehicle or anionic clodronate liposome (Clophosome®-A) 24 hours before the administration of control or engineered exosomes. Clophosome®-A composed of anionic lipids, which deplete more than 90% macrophages in spleen after a single intravenous injection^{24,25}. Clophosome®-A is not approved for human studies, and it is for experimental use only. Orthotopic breast cancer was developed by injecting 50,000 cells in the fat pad of right lower breast. Untreated animals were used as a positive control, and Clophosome®-A treated animal were used as negative control. 24 hours after the treatment (5 weeks old tumor-bearing animals), the mice were used to determine the accumulation of IV administered DiI labeled control and engineered exosomes in the tumors, spleen, liver and lungs. Three hours after IV administration of exosomes the organs were harvested with proper perfusion. Half of the tumors and organs including lymph nodes were fixed, and sectioned for immunohistochemical studies. Immunohistochemistry was conducted to determine the accumulation of DiI labeled exosomes in CD206+ and CD206− cells.

Immunofluorescent staining of frozen sections Harvested tissues (tumor, spleen and Lungs) from the animals were transferred to 30% sucrose and 3% paraformaldehyde solution. 10 μm thick sections were prepared and collected on to pre-warmed slides, and allowed to dry at least for a day. Sections were covered with ˜200-μL of blocking solution and were placed in the humidity box for 20-30 minutes at room temperature. Blocking solution was gently flicked away and appropriate primary antibodies diluted in blocking solution was added. The slides were incubated in humidity box overnight at 4° C. Then the slides were washed twice at least 5 minutes per wash. Secondary antibodies diluted in blocking solution was added to the sections and incubated at room temperature for two hours in humidity box or overnight at 4° C. Then the slides were washed twice at least 5 minutes per wash followed by counter stain with DAPI for nuclear stain. After final wash step, slides were mounted with ProLong™ Gold Antifade mounting media (Invitrogen™) and with an 18×18-1 glass coverslips.

### Western Blot

Cells and tissues were processed for protein isolation using Pierce RIPA buffer (Thermo Scientific, USA). Protein concentrations were estimated with Pierce, BCA protein assay kit (Thermo Scientific, USA), and separated by standard Tris/Glycine/SDS gel electrophoresis. Membranes were blocked with Odyssey Blocking buffer (LI-COR, Lincoln, Nebr.) for 60 min at room temperature and incubated with primary antibody against 6×His-tag (BioLegend, cat #362602, 1:500) antibody followed by horseradish peroxidase-conjugated secondary antibody (1:5000,). The blot was developed using a Pierce Super Signal West Pico Chemiluminescent substrate kit (Thermo Scientific, USA). Western blot images were acquired by Las-3000 imaging machine (Fuji Film, Japan).

### Use of Engineered Exosomes Carrying Fusion Protein as Therapeutic Probes

In vitro studies to assess phagocytosis and cytotoxicity using exosome-Fc-mIgG2b complex: We used CFSE-stained Raw264.7 converted to M2 macrophages using IL4 and IL-13 and MEF co-cultured with splenocytes at different ratios. Twenty-four hours after co-culture, engineered exosomes carrying Fc-mIgG2b were added to the co-culture, and the. The studies were repeated at least three times for reproducibility and there was multiple replicate at each time.

### Statistical Analysis

Quantitative data were expressed as mean±standard error of the mean (SEM) unless otherwise stated, and statistical differences between more than two groups were determined by analysis of variance (ANOVA) followed by multiple comparisons using Tukey's multiple comparisons test. Comparison between 2 samples was performed by Student t test. GraphPad Prism version 8.2.1 for Windows (GraphPad Software, Inc., San Diego, Calif.) was used to perform the statistical analysis. We used a significance level of 5% (α=0.05) and for a power of 80% (the chance of detecting a significant difference if there's any), the sample size required for the experiments were between 3 or 4 animals per group. The same sample size also was valid for a 90% power calculation. For this reason, we fixed our sample size to n=3 or n=4 as mentioned in the methodology. Differences with p-values less than 0.05 were considered significant (*p<0.05, **p<0.01, ***p<0.001, ****p<0.0001).

Plasmid Sequence
Plasmid final version (targeting + therapeutic)
NheI - Lamp2b signal peptide - linker - CD206 target
(TGCTCTCCGGGGGCGAAAGTCAGGTGC(SEW ID NO: 2)) - linker -
mIgG2b - linker - Lamp2b remaining sequence - His-tag -
stop codon - EcoRI
(SEQ ID NO: 3)
GCTAGCATGTGCCTCTCTCCGGTTAAAGGCGCAAAGCTCATCCTGATCTTTCTGTTCC
TAGGAGCCGTTCAGTCCAATGCAGCGCGATGCTCTCCGGGGGCGAAAGTCAGGTGC
GCTCGTGGCCCATTTCAACAATCAACCCCTGTCCTCCATGCAAGGAGTGTCACAAAT
GCCCAGCTCCTAACCTCGAGGGTGGACCATCCGTCTTCATCTTCCCTCCAAATATCA
AGGATGTACTCATGATCTCCCTGACACCCAAGGTCACGTGTGTGGTGGTGGATGTGA
GCGAGGATGACCCAGACGTCCAGATCAGCTGGTTTGTGAACAACGTGGAAGTACAC
ACAGCTCAGACACAAACCCATAGAGAGGATTACAACAGTACTATCCGGGTGGTCAG
CACCCTCCCCATCCAGCACCAGGACTGGATGAGTGGCAAGGAGTTCAAATGCAAGG
TCAACAACAAAGACCTCCCATCACCCATCGAGAGAACCATCTCAAAAATTAAAGGG
CTAGTCAGAGCTCCACAAGTATACATCTTGCCGCCACCAGCAGAGCAGTTGTCCAGG
AAAGATGTCAGTCTCACTTGCCTGGTCGTGGGCTTCAACCCTGGAGACATCAGTGTG
GAGTGGACCAGCAATGGGCATACAGAGGAGAACTACAAGGACACCGCACCAGTCCT
GGACTCTGACGGTTCTTACTTCATATACAGCAAGCTCGATATAAAAACAAGCAAGTG
GGAGAAAACAGATTCCTTCTCATGCAACGTGAGACACGAGGGTCTGAAAAATTACT
ACCTGAAGAAGACCATCTCCCGGTCTCCGGGTAAATGAGCTCAGCACCCACAAAGC
TAGCTGGAGCGGAGGCTTGATAGTTAATTTGACAGATTCAAAGGGTACTTGCCTTTA
TGCAGAATGGGAGATGAATTTCACAATAACATATGAAACTACAAACCAAACCAATA
AAACTATAACCATTGCAGTACCTGACAAGGCGACACACGATGGAAGCAGTTGTGGG
GATGACCGGAATAGTGCCAAAATAATGATACAATTTGGATTCGCT
GTCTCTTGGGCTGTGAATTTTACCAAGGAAGCATCTCATTATTCAATTCATGACATCG
TGCTTTCCTACA ACACTAGTGA TAGCACAGTA TTTCCTGGTG
CTGTAGCTAAAGGAGTTCAT ACTGTTAAAA ATCCTGAGAA TTTCAAAGTT
CCATTGGATG TCATCTTTAAGTGCAATAGT GTTTTAACTT ACAACCTGAC
TCCTGTCGTT CAGAAATATT GGGGTATTCACCTGCAAGCT TTTGTCCAAA
ATGGTACAGT GAGTAAAAAT GAACAAGTGT GTGAAGAAGACCAAACTCCC
ACCACTGTGG CACCCATCAT TCACACCACT GCCCCGTCGA
CTACAACTACACTCACTCCA ACTTCAACAC CCACTCCAAC TCCAACTCCA
ACTCCAACCG TTGGAAACTACAGCATTAGA AATGGCAATA CTACCTGTCT
GCTGGCTACC ATGGGGC TGC AGCTGAACATCACTGAGGAG AAGGTGCCTT
TCATTTTTAA CATCAACCCT GCCACAACCA ACTTCACCGGCAGCTGTCAA
CCTCAAAGTG CTCAACTTAG GCTGAACAAC AGCCAAATTA
AGTATCTTGACTTTATCTTT GCTGTGAAAA ATGAAAAACG GTTCTATCTG
AAGGAAGTGA ATGTCTACATGTATTTGGCT AATGGCTCAG CTTTCAACAT
TTCCAACAAG AACCTTAGCT TCTGGGATGCCCCTCTGGGA AGTTCTTATA
TGTGCAACAA AGAGCAGGTG CTTTCTGTGT CTAGAGCGTTTCAGATCAAC
ACCTTTAACC TAAAGGTGCA ACCTTTTAAT GTGACAAAAG
GACAGTATTCTACAGCTGAG GAATGTGCTG CTGACTCTGA CCTCAACTTT
CTTATTCCTG TTGCAGTGGGTGTGGCCTTG GGCTTCCTTA TAATTGCTGT
GTTTATATCT TACATGATTG GAAGACGGAAAAGTCGTACT GGTTATCAGT CTGTC
CAC CAC CAC CAC CAC CAC TAA GAATTC.
Lamp2b signal peptide -
(SEQ ID NO: 4)
GCTAGCATGTGCCTCTCTCCGGTTAAAGGCGCAAAGCTCATCCTGATCTTTCTGTTCC
TAGGAGCCGTTCAGTCCAATGCA.
Lamp2b remaining sequence
(SEQ ID NO: 5)
GCTAGCATGTGCCTCTCTCCGGTTAAAGGCGCAAAGCTCATCCTGATCTTTCTGTTCC
TAGGAGCCGTTCAGTCCAATGCAGCGCGATGCTCTCCGGGGGCGAAAGTCAGGTGC
GCTCGTGGCCCATTTCAACAATCAACCCCTGTCCTCCATGCAAGGAGTGTCACAAAT
GCCCAGCTCCTAACCTCGAGGGTGGACCATCCGTCTTCATCTTCCCTCCAAATATCA
AGGATGTACTCATGATCTCCCTGACACCCAAGGTCACGTGTGTGGTGGTGGATGTGA
GCGAGGATGACCCAGACGTCCAGATCAGCTGGTTTGTGAACAACGTGGAAGTACAC
ACAGCTCAGACACAAACCCATAGAGAGGATTACAACAGTACTATCCGGGTGGTCAG
CACCCTCCCCATCCAGCACCAGGACTGGATGAGTGGCAAGGAGTTCAAATGCAAGG
TCAACAACAAAGACCTCCCATCACCCATCGAGAGAACCATCTCAAAAATTAAAGGG
CTAGTCAGAGCTCCACAAGTATACATCTTGCCGCCACCAGCAGAGCAGTTGTCCAGG
AAAGATGTCAGTCTCACTTGCCTGGTCGTGGGCTTCAACCCTGGAGACATCAGTGTG
GAGTGGACCAGCAATGGGCATACAGAGGAGAACTACAAGGACACCGCACCAGTCCT
GGACTCTGACGGTTCTTACTTCATATACAGCAAGCTCGATATAAAAACAAGCAAGTG
GGAGAAAACAGATTCCTTCTCATGCAACGTGAGACACGAGGGTCTGAAAAATTACT
ACCTGAAGAAGACCATCTCCCGGTCTCCGGGTAAATGAGCTCAGCACCCACAAAGC
TAGCTGGAGCGGAGGCTTGATAGTTAATTTGACAGATTCAAAGGGTACTTGCCTTTA
TGCAGAATGGGAGATGAATTTCACAATAACATATGAAACTACAAACCAAACCAATA
AAACTATAACCATTGCAGTACCTGACAAGGCGACACACGATGGAAGCAGTTGTGGG
GATGACCGGAATAGTGCCAAAATAATGATACAATTTGGATTCGCT
GTCTCTTGGGCTGTGAATTTTACCAAGGAAGCATCTCATTATTCAATTCATGACATCG
TGCTTTCCTACA ACACTAGTGA TAGCACAGTA TTTCCTGGTG
CTGTAGCTAAAGGAGTTCAT ACTGTTAAAA ATC CTGAGAA TTTCAAAGTT
CCATTGGATG TCATCTTTAAGTGCAATAGT GTTTTAACTT ACAACCTGAC
TCCTGTCGTT CAGAAATATT GGGGTATTCACCTGCAAGCT TTTGTCCAAA
ATGGTACAGT GAGTAAAAAT GAACAAGTGT GTGAAGAAGACCAAACTCCC
ACCACTGTGG CACCCATCAT TCACACCACT GCCCCGTCGA
CTACAACTACACTCACTCCA ACTTCAACAC CCACTCCAAC TCCAACTCCA
ACTCCAACCG TTGGAAACTACAGCATTAGA AATGGCAATA CTACCTGTCT
GCTGGCTACC ATGGGGC TGC AGCTGAACATCACTGAGGAG AAGGTGCCTT
TCATTTTTAA CATCAACCCT GCCACAACCA ACTTCACCGGCAGCTGTCAA
CCTCAAAGTG CTCAACTTAG GCTGAACAAC AGCCAAATTA
AGTATCTTGACTTTATCTTT GCTGTGAAAA ATGAAAAACG GTTCTATCTG
AAGGAAGTGA ATGTCTACATGTATTTGGCT AATGGCTCAG CTTTCAACAT
TTCCAACAAG AACCTTAGCT TCTGGGATGCCCCTCTGGGA AGTTCTTATA
TGTGCAACAA AGAGCAGGTG CTTTCTGTGT CTAGAGCGTTTCAGATCAAC
ACCTTTAACC TAAAGGTGCA ACCTTTTAAT GTGACAAAAG
GACAGTATTCTACAGCTGAG GAATGTGCTG CTGACTCTGA CCTCAACTTT
CTTATTCCTG TTGCAGTGGGTGTGGCCTTG GGCTTCCTTA TAATTGCTGT
GTTTATATCT TACATGATTG GAAGACGGAAAAGTCGTACT GGTTATCAGT CTGTC.
CD206 target
(SEQ ID NO: 2)
TGCTCTCCGGGGGCGAAAGTCAGGTGC
mIgG2b
(SEQ ID NO: 6)
GGCCCATTTCAACAATCAACCCCTGTCCTCCATGCAAGGAGTGTCACAAATGCCCAG
CTCCTAACCTCGAGGGTGGACCATCCGTCTTCATCTTCCCTCCAAATATCAAGGATG
TACTCATGATCTCCCTGACACCCAAGGTCACGTGTGTGGTGGTGGATGTGAGCGAGG
ATGACCCAGACGTCCAGATCAGCTGGTTTGTGAACAACGTGGAAGTACACACAGCT
CAGACACAAACCCATAGAGAGGATTACAACAGTACTATCCGGGTGGTCAGCACCCT
CCCCATCCAGCACCAGGACTGGATGAGTGGCAAGGAGTTCAAATGCAAGGTCAACA
ACAAAGACCTCCCATCACCCATCGAGAGAACCATCTCAAAAATTAAAGGGCTAGTC
AGAGCTCCACAAGTATACATCTTGCCGCCACCAGCAGAGCAGTTGTCCAGGAAAGA
TGTCAGTCTCACTTGCCTGGTCGTGGGCTTCAACCCTGGAGACATCAGTGTGGAGTG
GACCAGCAATGGGCATACAGAGGAGAACTACAAGGACACCGCACCAGTCCTGGACT
CTGACGGTTCTTACTTCATATACAGCAAGCTCGATATAAAAACAAGCAAGTGGGAG
AAAACAGATTCCTTCTCATGCAACGTGAGACACGAGGGTCTGAAAAATTACTACCTG
AAGAAGACCATCTCCCGGTCTCCGGGTAAATGAGCTCAGCACCCACAAAGCTAGCT
GG.

### EXAMPLES

### Example 1: Determination of Specificity of Precision Peptide In Vivo and Generation of CD206-Positive M2 Macrophage-Specific Exosomes

Methods and Materials

To assess in vivo targeting potential, rhodamine-labeled precision peptide (red) was injected intravenously (IV) in metastatic syngeneic murine breast cancer (4T1) bearing Balb/C mice. Three hours after injection, all animals were euthanized, and lungs, spleen and tumors were collected for immune-histochemical analysis. Frozen sections from the collected tissues were stained for CD206 (fluorescein, FITC) and counter stained with DAPI. To confer targeting potentiality, precision peptide for CD206-positive TAMs was fused to the extra-exosomal N-terminus of murine Lamp2b.

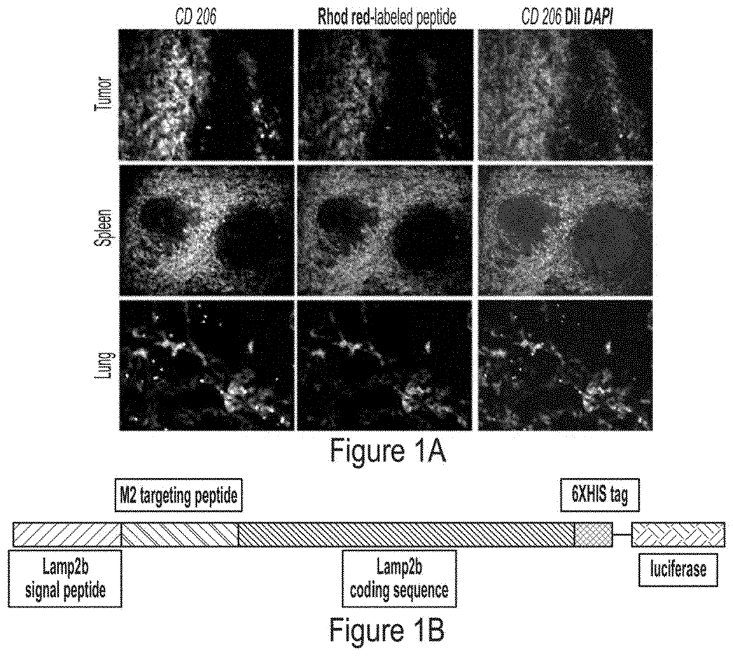

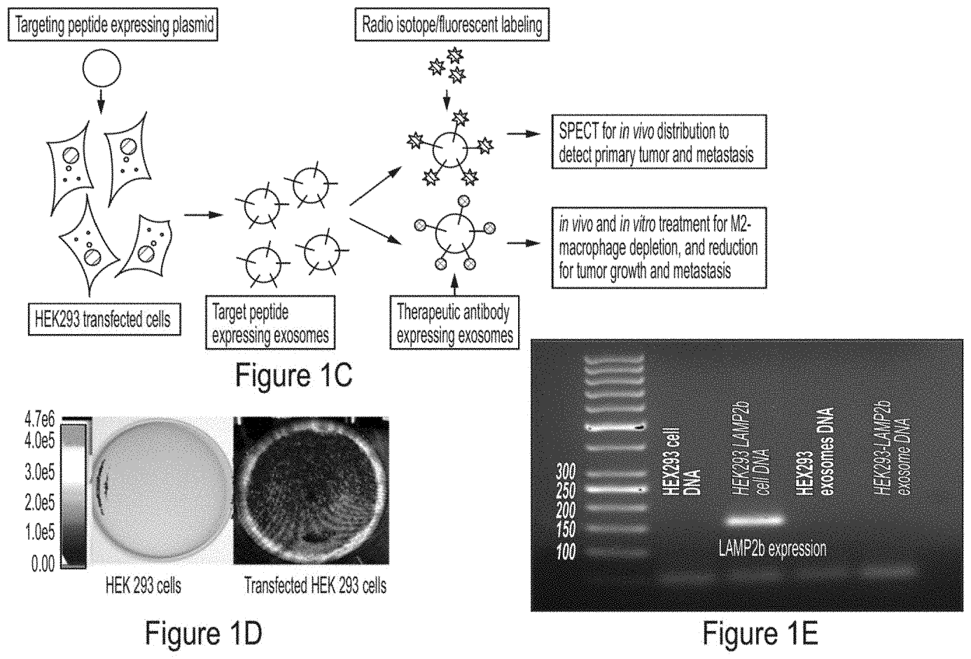

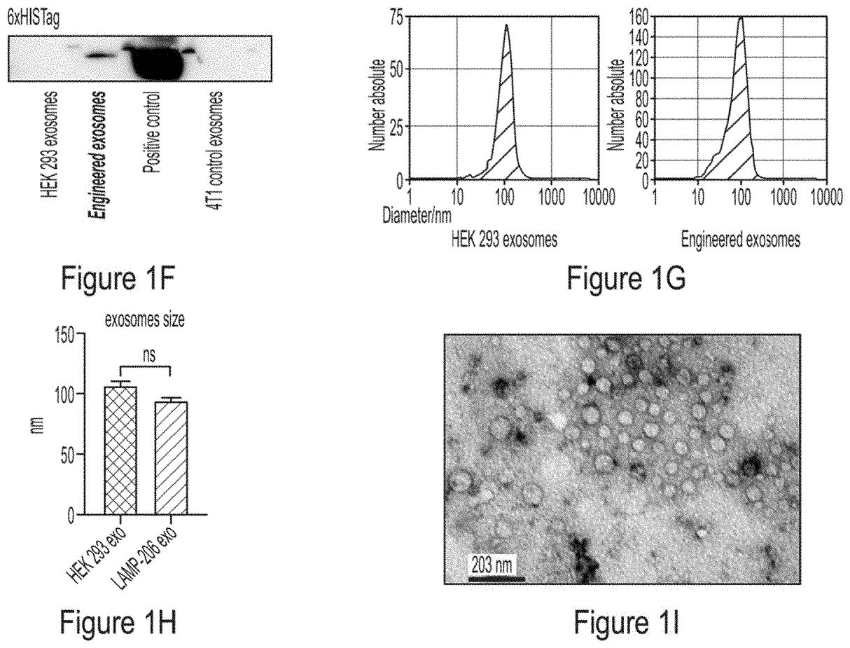

Results FIG. 1 represents generation of engineered exosomes expressing CD206-positive M2 macrophage-specific peptide along with Lamp2b. FIG. 1a exhibits immunofluorescence staining of tumor, spleen and lungs sections from 4T1 tumor-bearing mice showing co-localization of Rhodamine red-labeled targeting peptide (injected i.v.) and FITC labeled CD206-positive M2-macrophages. Nuclei were visualized by DAPI staining (blue). FIG. 1b is a schematic representation of the modified Lamp2b protein containing CD206 positive M2 macrophage-targeting peptide sequence following signal peptide, and a 6×HIS tag at the C terminus. Luciferase was used as a reporter gene. FIG. 1c is a schematic diagram showing generation of CD206+M2-macrophage targeting engineered exosomes for diagnostic and therapeutic purpose. FIG. 1d represents in vitro study showing luciferase activity of transfected HEK293 cells. FIG. 1e is agarose gel electrophoresis showing confirmation of targeting peptide sequence insert in transfected HEK293 cells. Figure if is a Western blot image showing anti-His tag antibody positivity in engineered exosomal protein content. FIG. 1g and FIG. 1h showing size distribution by nanoparticle tracking assay (NTA) of the HEK293 exosomes and engineered exosomes, respectively. Quantitative data are expressed in mean±SEM. FIG. 1i illustrates a transmission electron microscopy image for engineered exosomes, (Scale bar depicts 200 nm) showing characteristic round morphology and size without any deformity.

### Example 2: Targeting Potential of CD206-Positive M2-Macrophage-Specific Exosomes

Methods and Materials

To assess targeting ability of the engineered exosomes, mouse RAW264.7 macrophages towards M2-macrophages was differentiated by treating them with IL-4 and IL-3 in vitro. The cells were co-cultured with DiI-labeled (red) engineered exosomes for 4 hours followed by immunofluorescence staining for CD206-positive cells (FITC) and DAPI for nuclei.

Results

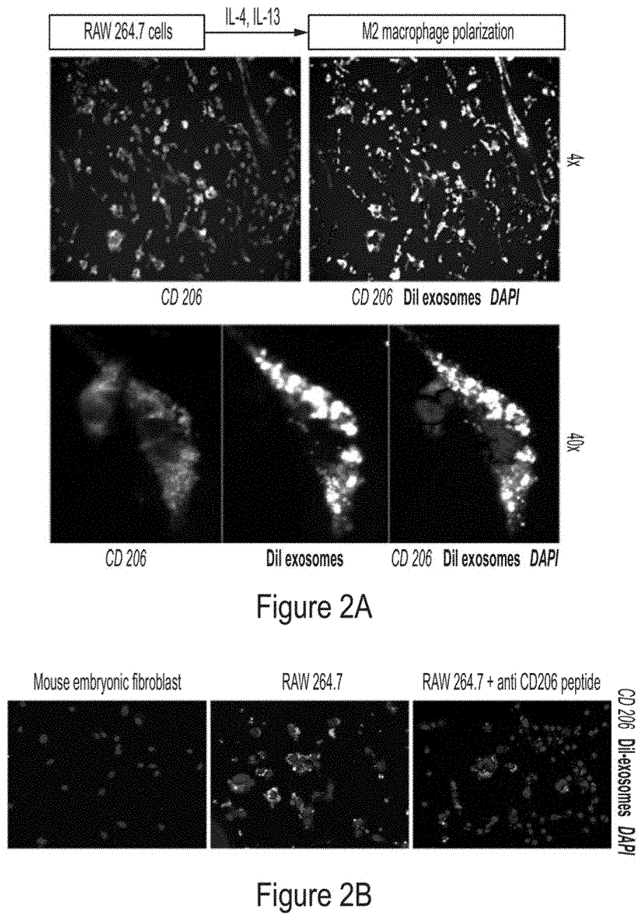

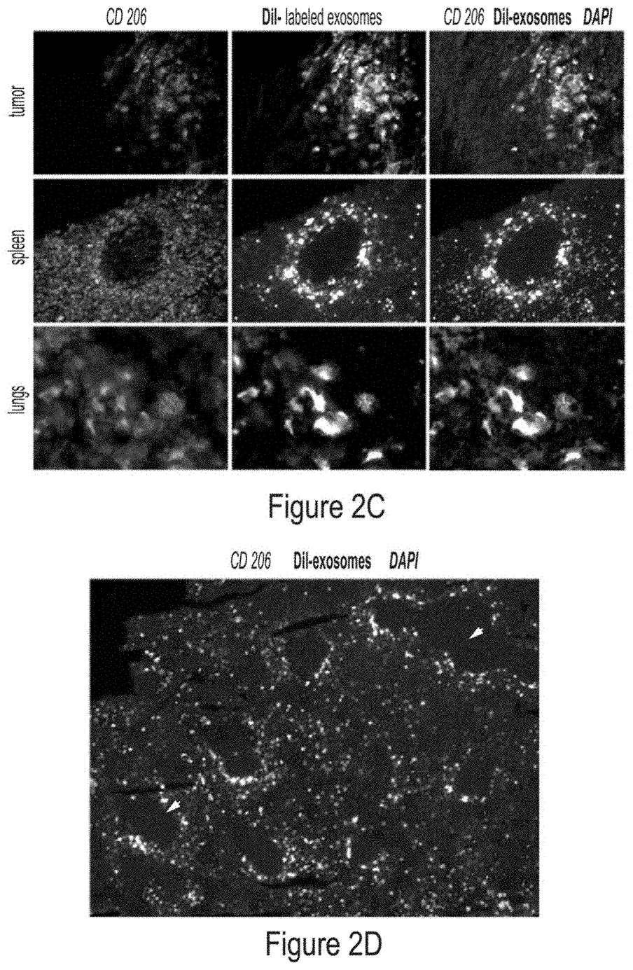

FIG. 2 represents targeting efficiency and specificity of CD206-positive M2 macrophage-specific exosomes. FIG. 2a exhibits immunofluorescence staining showing targeting potential of DiI-labeled (red) engineered exosomes. RAW264.7 mouse macrophages were differentiated to CD206-positive (FITC) cells by treating with interleukin-4 and interleukin-13. Nuclei were visualized by DAPI staining (blue). FIG. 2b exhibits immunofluorescence staining of mouse embryonic fibroblasts (MEFs) and RAW264.7 cells treated with or without anti-CD206 peptide, co-cultured with DiI-labeled (red) engineered exosomes. MEFs were negative for CD206 (FITC) staining and did not take up the exosomes. Engineered exosomes bound to the CD206+ RAW264.7 cells, that was prevented by anti-CD206 peptide treatment. FIG. 2c exhibits immunofluorescence staining of tumor, spleen and lungs sections from 4T1 tumor-bearing mice showing co-localization of rhodamine red-labeled targeting exosomes (injected i.v.) and FITC labeled CD206-positive M2-macrophages. Nuclei were visualized by DAPI staining (blue). FIG. 2d exhibits stitched images for extended view of splenic section showing engineered exosomes were not taken up by T-lymphocytes and B-lymphocytes in splenic white pulp (white arrows).

### Example 3: Detection and Quantification of In Vivo Distribution of CD206-Positive M2 Macrophages Targeting Exosomes

Methods and Materials

To investigate the validity of engineered exosomes as an imaging probe to determine the distribution of M2-macrophages, FDA approved clinically relevant SPECT scanning and labeling with ¹¹¹In-oxine was used according to our previous study (Arbab et al., BMC Med. Imaging 2012, 12, 33). ¹¹¹In-oxine-labeled non-engineered control exosomes (HEK293 exo) in metastatic (4T1) mouse breast cancer models, and engineered exosomes (M2-targeting exo) expressing precision peptide treated with either vehicle or clodronate liposome (Clophosome®-A) 24 hours before the IV administration of ¹¹¹In-oxine-labeled exosomes and SPECT studies was used. Clophosome®-A is composed of anionic lipids and depletes more than 90% macrophages in spleen after a single intravenous injection (Li et al., Scient. Rep. 2016, 6, 22143-22143; Kobayashi et al., J. Biol. Chem. 2015, 290, 12603-12613). Clophosome®-A is not approved for human studies, and it is for experimental use only. Similar to the previously-mentioned, ¹³¹I-labeled exosomes (Rashid et al., Nanomed: Nanotechnol., Biol. Med. 2019, 21, 102072), prior to IV injection into mice for biodistribution, the labeling efficiency of ¹¹¹In-oxine to the engineered exosomes and serum stability of binding by thin layer paper chromatography (TLPC) was checked.

Results

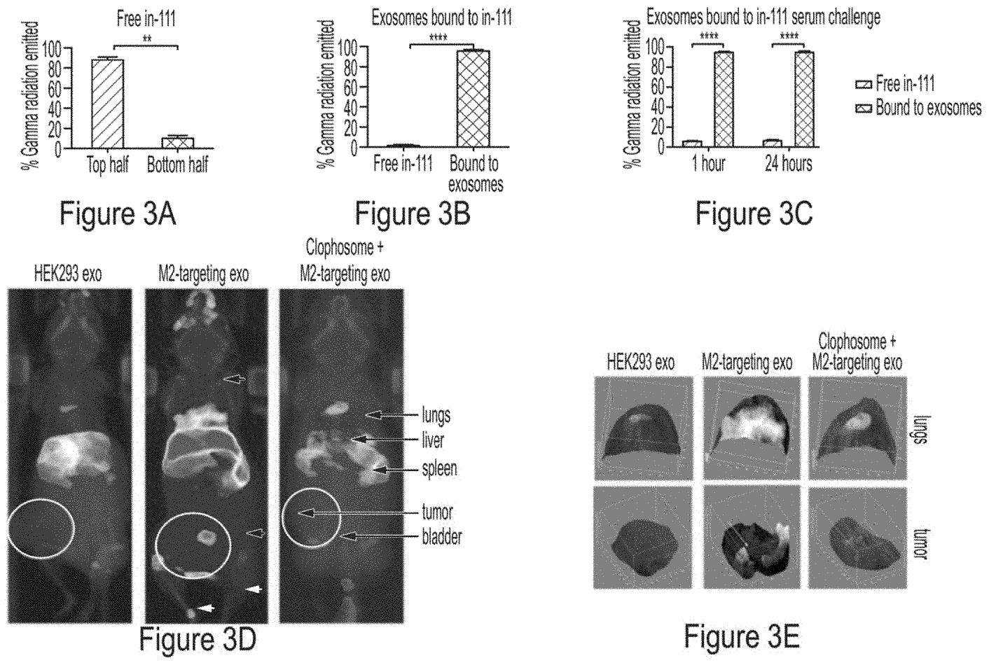

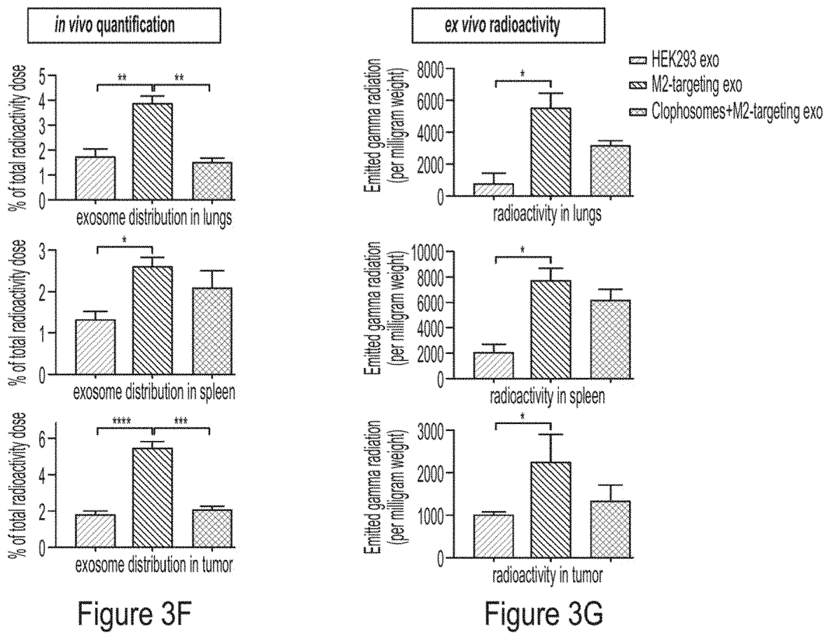

FIG. 3 represents detection and quantification of biodistribution of ¹¹¹In-oxine-labeled exosomes targeting CD206-positive M2 macrophages. FIG. 3a shows a major proportion of the free ¹¹¹In-oxine measured in the bottom to the top half of the thin layer paper chromatography (TLPC) paper, confirming the efficacy of the eluent. FIG. 3b shows binding of ¹¹¹In-oxine to engineered exosomes was validated as shown by a lower percentage of In-oxine (free, dissociated) measured in the top of the paper, compared to the amount remaining in the bottom, which represented the ¹¹¹In-oxine-labeled exosomes. FIG. 3c shows serum stability of ¹¹¹In-oxine bound engineered exosomes was higher compared with the small amount of free ¹¹¹In-oxine disengaged from the bound exosomes. FIG. 3d illustrates in vivo SPECT/CT images (coronal view) after 3 hrs of intravenous injection showed significant accumulation of M2-targeting exo in tumor, lung, spleen, lymph node and bones. ¹¹¹In-oxine-labeled non-targeting exosomes (HEK293 exo) and CD206-positive M2-macrophage targeting exosomes (M2-targeting exo) were injected into the 4T1 tumor-bearing mice. One group was treated with Clophosome® to deplete macrophages. Yellow and green arrows denote lymph node and bone metastasis, respectively. FIG. 3e illustrates 3D surface images showing M2-targeting exo are profoundly distributed in both lung and tumor area compared to the group injected with HEK293 exo and pre-treated with Clophosome®. Yellow arrow indicates the tumor center. FIG. 3f shows quantification of in vivo radioactivity in lungs, spleen and tumor. FIG. 3g shows ex vivo radioactivity quantification in lungs, spleen and tumor. Quantitative data are expressed in mean±SEM. *P<0.05, **P<0.01, ***P<0.001, ****P<0.0001. n=3.

### Example 4: Generation of CD206-Positive M2 Macrophage-Targeting Therapeutic Exosomes

Methods and Materials

Following the confirmation of targeting potential of engineered exosomes for diagnostic purpose, the exosomes as therapeutic carriers was further utilized. Fc portion of mouse IgG2b next to the targeting precision peptide with a small linker with the purpose of inducing ADCC was conjugated. 6×His tag and luciferase were incorporated as reporter genes similar to the previous construct.

Results

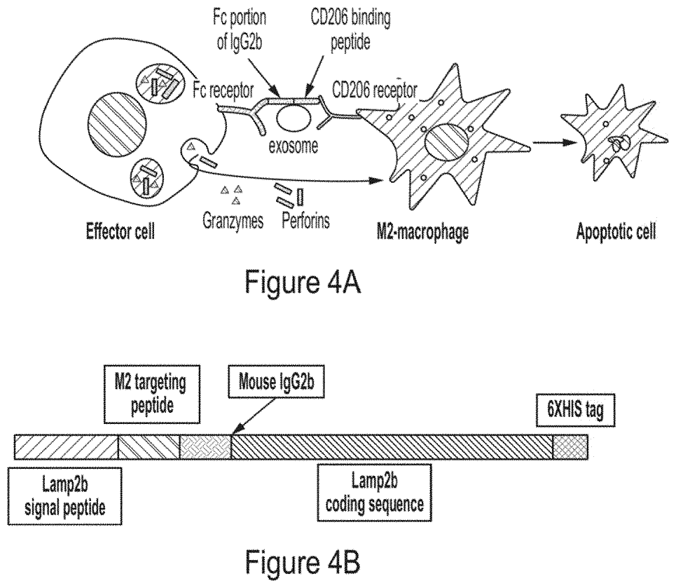

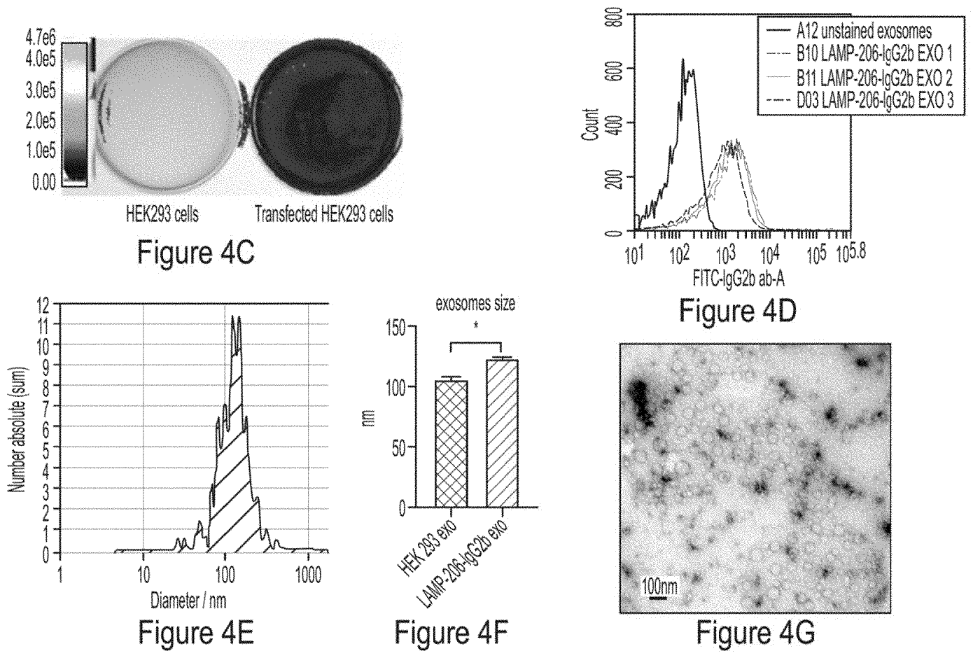

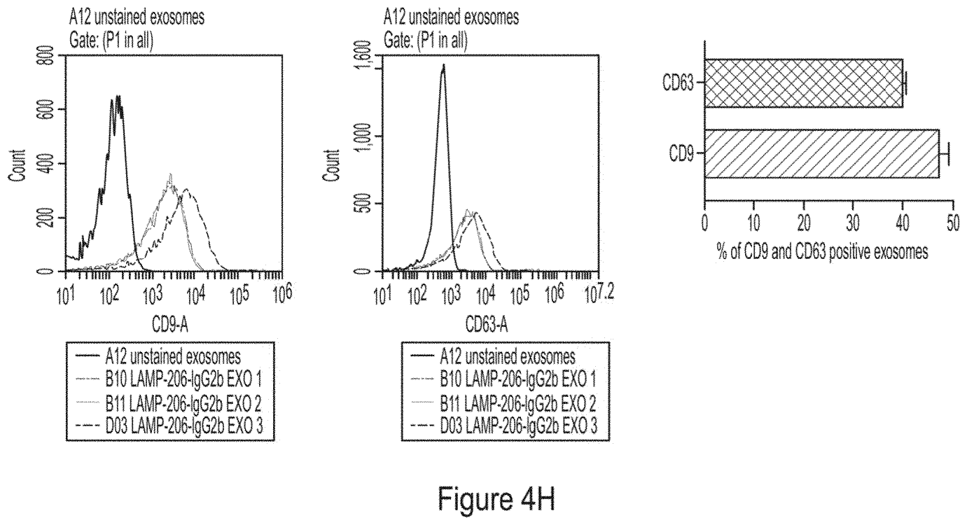

FIG. 4 represents generation of CD206-positive M2 macrophage-targeting therapeutic exosomes to induce antibody-dependent cell-mediated cytotoxicity. FIG. 4a illustrates schematic diagram showing the proposed mechanism of engineered exosome-based antibody-dependent cellular cytotoxicity. FIG. 4b illustrates schematic representation of the plasmid construct containing modified Lamp2b protein with CD206-targeting sequence conjugated with Fc segment of mouse IgG2b. FIG. 4c demonstrates confirmation of luciferase activity by transfected HEK293 cells. FIG. 4d shows flow cytometry analysis for validating the expression of Fc segment of mouse IgG2b on the surface of engineered exosomes. Three different engineered exosome samples were used for the flow cytometry. FIG. 4e shows concentration and size distribution of the engineered therapeutic exosomes by nanoparticle tracking assay (NTA). FIG. 4f shows mean diameter of engineered exosomes was significantly larger than non-engineered exosomes. FIG. 4g illustrates transmission electron microscopy image for engineered therapeutic exosomes, (Scale bar depicts 100 nm) showing distinctive round morphology and size without any distortion. FIG. 4h shows flow-cytometry analysis of exosomal markers CD9 and CD63 for the engineered therapeutic exosomes. Three different engineered exosome samples were used for the flow cytometry.

### Example 5: Induction of Cytotoxicity and Depletion of M2-Macrophages by Engineered Therapeutic Exosomes

Methods and Materials

To ascertain the capacity of therapeutic exosomes for instigating ADCC, the CFSE-labeled (green) RAW264.7 macrophages was treated with non-therapeutic CD206-positive cell-targeting exosomes (LAMP-206 exo) or CD206-positive cell-targeting therapeutic exosomes (LAMP-206-IgG2b exo), and without any exosome treatment (control) for 48 hours in presence of normal mouse splenic mononuclear cells.

Results

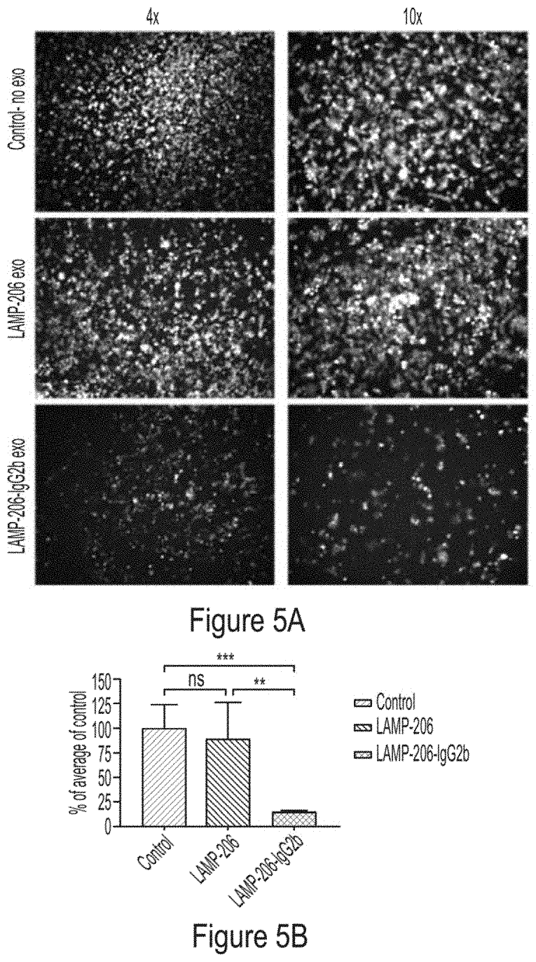

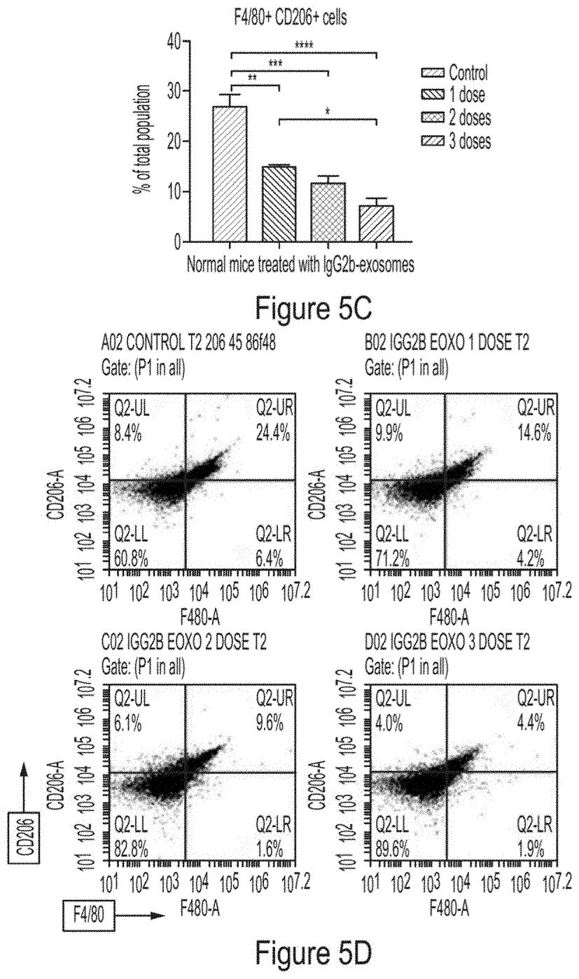

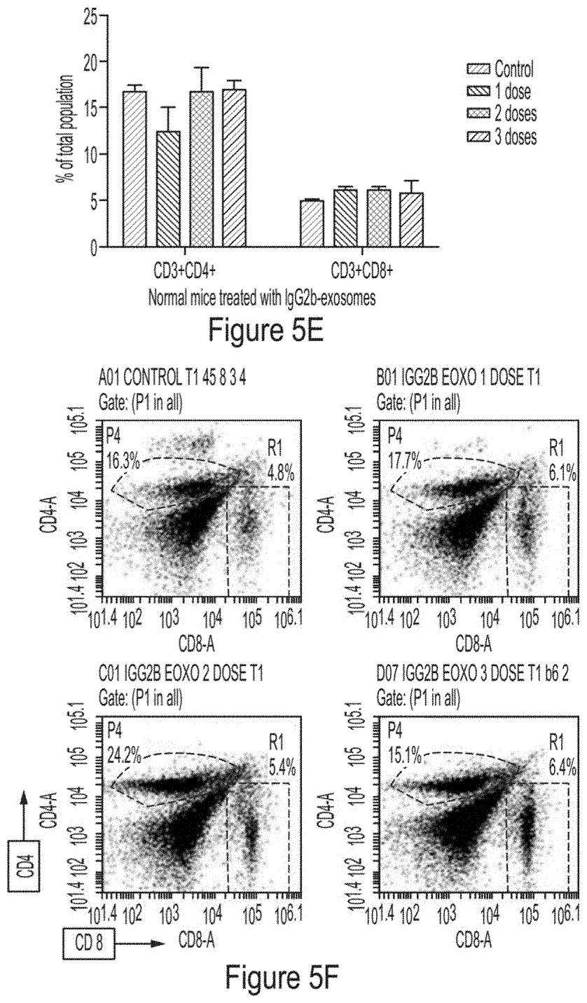

FIG. 5 represents therapeutic efficiency and specificity of engineered therapeutic exosomes in depleting M2-macrophages both in vitro and in vivo. FIG. 5a illustrates CFSE-labeled (green) RAW264.7 mouse macrophages were co-cultured with non-therapeutic CD206-positive cell-targeting exosomes (LAMP-206 exo) or CD206-positive cell-targeting therapeutic exosomes (LAMP-206-IgG2b exo), and without treatment (control) for 48 hours in presence of splenic immune cells from normal mice. Fluorescence microscopic images showed decrease in cell number and increased floating dead cells in LAMP-206-IgG2b exo group compared to other groups. FIG. 5b shows measured fluorescence intensity of the above-mentioned conditions showed significant decrease in LAMP-206-IgG2b exo group compared to other groups. FIG. 5c and FIG. 5d exhibits normal Balb/c mice were treated with one, two or three doses of engineered therapeutic exosomes expressing Fc portion of mouse IgG2b. Flow-cytometry analysis of splenic cells showing dose-dependent decline of F4/80 and CD206-positive M2-macrophage population. FIG. 5e and FIG. 5f illustrates flow-cytometry analysis of splenic cells showing no significant change in both CD4 and CD8-positive T-cell population after treating the mice with different doses of therapeutic exosomes. Quantitative data are expressed in mean±SEM. *P<0.05, **P<0.01, ***P<0.001, ****P<0.0001. n=5.

### Example 6: Treatment with Engineered Therapeutic Exosomes Prevent Tumor Growth and Early Metastasis Increasing Survival

Methods and Materials

Furthermore, in vivo distribution of the precision peptide after therapeutic exosome treatment in mouse tumor model to see if the treatment can attenuate distribution of the peptide in M2-macrophage prevalent areas was determined. Tumor cells were implanted subcutaneously on the flanks of mice. After 3 weeks of tumor growth, one group of mice was treated with engineered therapeutic exosomes for one week (3 doses), and another group of mice was without treatment. 6-Hydrazinopyridine-3-carboxylic acid (HYNIC) was conjugated with the precision peptide and labeled with technetium-99m (99mTc). 99mTc-labeled peptide was injected into both groups of mice and after 3 hours CT followed by SPECT images were acquired.

Results

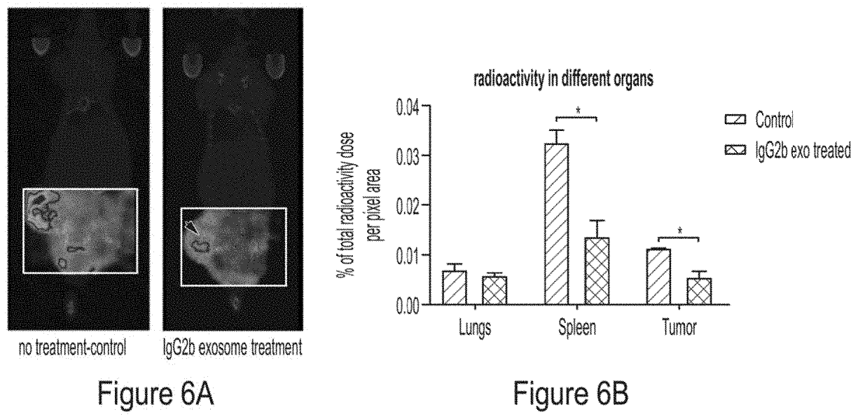

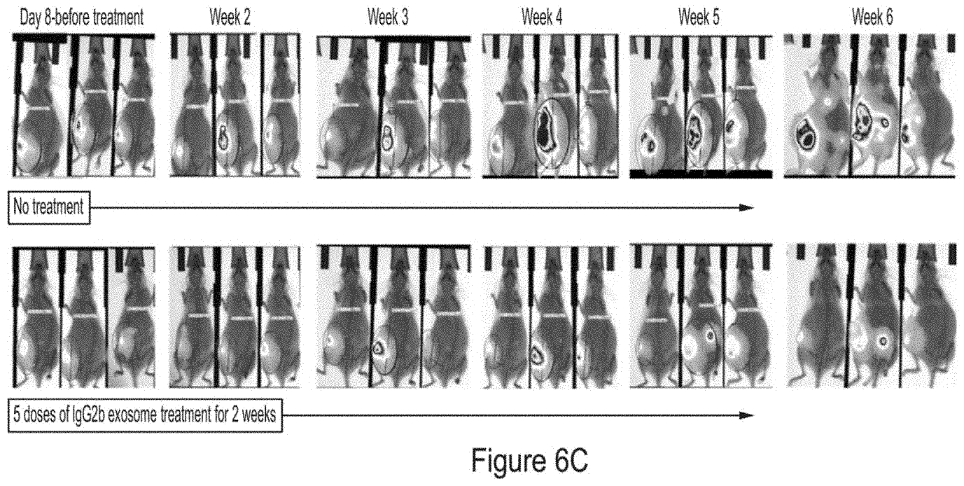

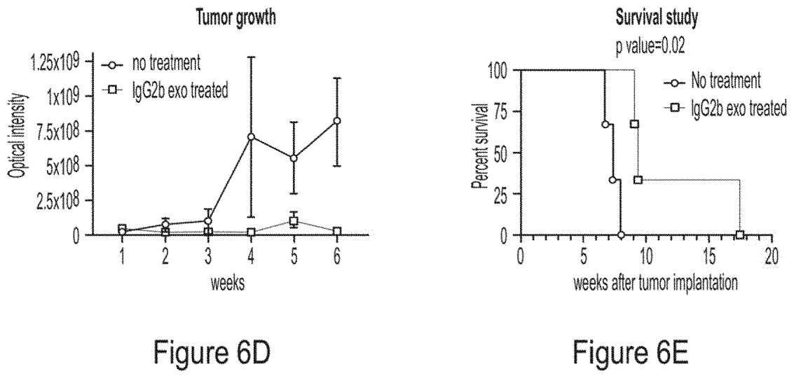

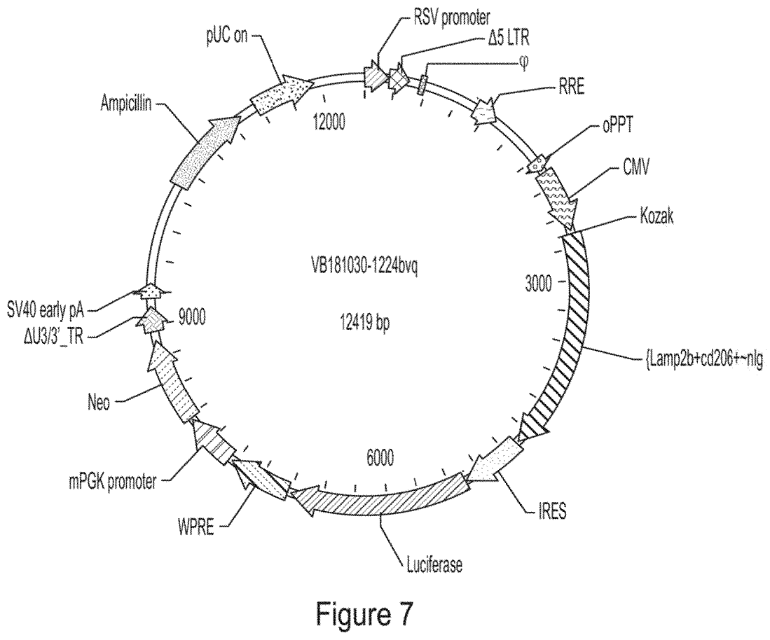

FIG. 6 represents treatment of 4T1 tumor-bearing animals with therapeutic engineered exosomes prevent tumor growth and metastasis, and improve survival by depleting M2-macrophages. FIG. 6a and FIG. 6b illustrates reconstructed and co-registered in vivo SPECT/CT images (coronal view) and quantification of subcutaneous syngeneic tumor-bearing animals (on the flank) injected with the 99mTc-labeled precision peptide after three hours. Group treated with therapeutic exosomes showed lesser level of radioactivity in tumor (yellow arrow) and spleen compared to untreated control group. Quantitative data are expressed in mean±SEM, *P<0.05. n=3. FIG. 6c displays optical images of 4T1 tumor-bearing animals treated with engineered therapeutic exosomes (lower panel) or without treatment (control), showing decreased tumor growth in treated animals compared to control group. Metastatic foci in control group was detected (yellow arrows) as early as fourth week, whereas no metastasis was detected in treated animals after 6 weeks. FIG. 6d illustrates quantification of optical density of the tumor area also showed decreased tumor growth in treated group compared to control group. Quantitative data are expressed in mean±SEM. n=3. FIG. 6e shows Kaplan-Meier plot showing prolonged survival of the mice treated with therapeutic engineered exosomes.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A CD206-positive M2 macrophage-targeting exosome expressing a CD206 binding peptide and an Fc portion of IgG2b.

2. The exosome of claim 1, wherein the CD206 binding peptide is encoded by a nucleic acid sequence having 95%, 99%, or 100% sequence identity to SEQ ID NO:2 and the IgG2b is encoded by a sequence having 95%, 99%, or 100% sequence identity to SEQ ID NO:6.

3. A vector encoded by a nucleic acid sequence having 85%, 90%, 95%, or 100% to SEQ ID NO:5.

4. A method for making CD206-positive M2 macrophage-targeting exosomes comprising:
transfecting macrophage with the vector of claim 3;
culturing the transfected macrophage in the presence of IL4 and IL-3; and
harvesting the CD206-positive M2 macrophage-targeting exosomes.

5. The method of claim 4, wherein the cells are RAW264.7macrophage cells.

6. The CD206-positive M2 macrophage-targeting exosomes of claim 1 or 2, wherein the CD206-positive M2 macrophage-targeting exosomes are loaded with cargo.

7. The CD206-positive M2 macrophage-targeting exosomes of claim 6, wherein the cargo is selected from the group consisting of a detectable label, a chemotherapeutic agent, and a cytotoxic agent.

8. A pharmaceutical composition comprising:
the CD206-positive M2 macrophage-targeting exosomes of claim 1 or 2; and
a pharmaceutically acceptable excipient.

9. A method of depleting M2 macrophage in a subject in need thereof, comprising:
administering an effective amount of the composition of claim 8 to the subject to deplete M2 macrophage in the subject.

10. The method of claim 9, wherein the subject is human.

11. The method of claim 10, wherein the subject has cancer.

12. The method of claim 11, wherein the cancer is metastatic breast cancer.

13. A method for treating cancer in a subject in need thereof comprising:
administering an effective amount of the composition of claim 8 to the subject to deplete pro-tumorigenic macrophage in the subject.

14. A method of reducing tumor burden in a subject in need thereof comprising:
administering an effective amount of the composition of claim 8 to the subject to reduce tumor burden in the subject.

15. A method for inducing antibody-dependent cell-mediated cytotoxicity in a subject in need thereof comprising:
administering an effective amount of the composition of claim 8 to the subject to induce antibody-dependent cell-mediated cytotoxicity in the subject.

16. A method for detecting cancer cells comprising
contacting a biological sample with the CD206-positive M2 macrophage-targeting exosomes of claim 7,
detecting the detectable label, wherein the detection of the label indicates the presence of cancer cells.

17. The method of claim 13, wherein the pro-tumorigenic macrophage is a M2 macrophage.
